(19) United States
(12) Patent Application Publication
(10) Pub. No.: US 2006/0094763 A1
(43) Pub. Date: May 4, 2006

(54) Polymorphic forms of 6-[2-(methylcarbomoyl) phenyl sulfanyl]-3-E-[2-(pyridin-2-yl)ethenyl]indazole

(75) Inventors: Qiang Ye, San Diego, CA (US); Ryan Marshal Hart, San Diego, CA (US); Robert Kania, San Diego, CA (US); Michael Ouellette, San Diego, CA (US); Zhen Ping Wu, La Jolla, CA (US); Scott Edward Zook, San Diego, CA (US)

Correspondence Address: AGOURON PHARMACEUTICALS, INC., 10777 SCIENCE CENTER DRIVE, SAN DIEGO, CA, 92121, US

(73) Assignee: Agouron Pharmaceuticals, Inc.

(21) Appl. No.: 11/264,493

(22) Filed: Oct. 31, 2005

Related U.S. Application Data
(60) Provisional application No. 60/624,665, filed on Nov. 2, 2004.

(51) Int. Cl. A61K 31/4439; C07D 403/02

(52) U.S. Cl. 514/338; 546/275.7

(57) ABSTRACT

The present invention relates to novel polymorphic forms of 6-[2-(methylcarbamoyl)phenylsulfanyl]-3-E-[2-(pyridin-2-yl)ethenyl]indazole, and to processes for their preparation. Such polymorphic forms may be a component of a pharmaceutical composition and may be used to treat a hyperproliferative disorder or a mammalian disease condition mediated by protein kinase activity.

This application claims priority to U.S. Provisional Application No. 60/624,665 filed on Nov. 2, 2004, which is incorporated herein by reference in its entirety

### FIELD OF THE INVENTION

The present invention relates to novel polymorphic forms of 6-[2-(methylcarbamoyl)phenylsulfanyl]-3-E-[2-(pyridin-2-yl)ethenyl]indazole and to methods for their preparation. The invention is also directed to pharmaceutical compositions containing at least one polymorphic form and to the therapeutic or prophylactic use of such polymorphic forms and compositions.

### BACKGROUND OF THE INVENTION

The compound 6-[2-(methylcarbamoyl)phenylsulfanyl]-3-E-[2-(pyridin-2-yl)ethenyl]indazole (also referred to as “Compound 1”), 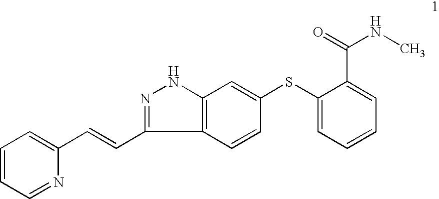
as well as pharmaceutically acceptable salts thereof, are described in U.S. Pat. No. 6,534,524, issued Mar. 18, 2003 and U.S. Pat. No. 6,531,491, issued Mar. 11, 2003, the disclosures of which are hereby incorporated in their entireties by reference for all purposes. This compound is a protein kinase receptor inhibitor and represents a synthetic, small molecule inhibitor of angiogenic receptor signaling.

Protein kinases are a family of enzymes that catalyze phosphorylation of the hydroxyl group of specific tyrosine, serine, or threonine residues in proteins. Typically, such phosphorylation dramatically perturbs the function of the protein, and thus protein kinases are pivotal in the regulation of a wide variety of cellular processes, including metabolism, cell proliferation, cell differentiation, and cell survival. Of the many different cellular functions in which the activity of protein kinases is known to be required, some processes represent attractive targets for therapeutic intervention for certain disease states. Two examples are angiogenesis and cell-cycle control, in which protein kinases play a pivotal role.

Unwanted angiogenesis is a hallmark of several diseases, such as retinopathies, psoriasis, rheumatoid arthritis, age-related macular degeneration (AMD), and cancer (including solid tumors) Folkman, Nature Med., 1, 27-31 (1995). Protein kinases that have been shown to be involved in the angiogenic process include VEGF-R2 (vascular endothelial growth factor receptor 2, also known as KDR (kinase insert domain receptor) and as FLK-1). Thus, direct inhibition of the kinase activity of VEGF-R2 may result in the reduction of angiogenesis even in the presence of exogenous VEGF (see Strawn et al., Cancer Research, 56, 3540-3545 (1996)).

There is thus a need for effective inhibitors of protein kinases. Moreover, as is understood by those skilled in the art, it is desirable for kinase inhibitors to possess physical properties amenable to reliable formulation. These properties include stability to heat, moisture, and light.

Crystalline polymorphs are different crystalline forms of the same compound. The term polymorph may or may not include other solid state molecular forms including hydrates (e.g., bound water present in the crystalline structure) and solvates (e.g., bound solvents other than water) of the same compound. Different crystalline polymorphs have different crystal structures due to a different packing of the molecules in the lattice. This results in a different crystal symmetry and/or unit cell parameters which directly influences its physical properties such the X-ray diffraction characteristics of crystals or powders. A different polymorph, for example, will in general diffract at a different set of angles and will give different values for the intensities. Therefore X-ray powder diffraction can be used to identify different polymorphs, or a solid form that comprises more than one polymorph, in a reproducible and reliable way.

Crystalline polymorphic forms are of interest to the pharmaceutical industry and especially to those involved in the development of suitable dosage forms. If the polymorphic form is not held constant during clinical or stability studies, the exact dosage form used or studied may not be comparable from one lot to another. It is also desirable to have processes for producing a compound with the selected polymorphic form in high purity when the compound is used in clinical studies or commercial products since impurities present may produce undesired toxicological effects. Certain polymorphic forms may exhibit enhanced thermodynamic stability or may be more readily manufactured in high purity in large quantities, and thus are more suitable for inclusion in pharmaceutical formulations. Certain polymorphs may display other advantageous physical properties such as lack of hygroscopic tendencies, improved solubility, and enhanced rates of dissolution due to different lattice energies.

The discussion of the background to the invention herein is included to explain the context of the present invention. This is not to be taken as an admission that any of the material referred to was published, known, or part of the common general knowledge in any country as of the priority date of any of the claims.

### SUMMARY OF THE INVENTION

The present invention relates to novel polymorphic forms of 6-[2-(methylcarbamoyl)phenylsulfanyl]-3-E-[2-(pyridin-2-yl)ethenyl]indazole (also referred to as “Compound 1”). 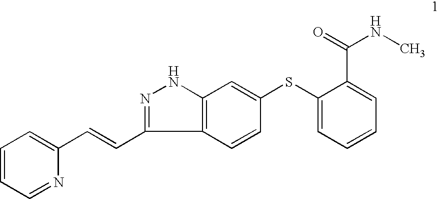
Compound 1 is a potent inhibitor of VEGF-R2 and has shown very favorable toxicological and pharmacological profiles. The present invention also relates to methods of preparing distinct polymorphic forms of Compound 1, their use in pharmaceutical compositions, and their use in the treatment of disease states associated with unwanted angiogenesis and/or cellular proliferation.

In one embodiment, the present invention provides a crystalline form of Compound 1, or a pharmaceutically acceptable salt thereof.

In another embodiment, the invention provides a crystalline form of Compound 1, or a pharmaceutically acceptable salt thereof, wherein the crystalline form is a polymorph designated as Form I. In a further embodiment, the invention provides a crystalline form of Compound 1, or a pharmaceutically acceptable salt thereof, wherein the crystalline form is a substantially pure polymorph of Form I. In a further embodiment, the invention provides a crystalline form of Compound 1, or a pharmaceutically acceptable salt thereof, that has a powder X-ray diffraction (PXRD) pattern comprising peaks at diffraction angles (2θ) of about 8.1 and about 29.8. Even more particularly, the invention provides a crystalline form of Compound 1, or a pharmaceutically acceptable salt thereof, that has a PXRD pattern comprising peaks at diffraction angles (2θ) of 8.1±0.1 and 29.8±0.1. Even more particularly, the invention provides a crystalline form of Compound 1, or a pharmaceutically acceptable salt thereof, that has a PXRD pattern comprising peaks at diffraction angles (2θ) of about 8.1, about 18.2, about 18.5, and about 29.8. Even more particularly, the invention provides a crystalline form of Compound 1, or a pharmaceutically acceptable salt thereof, that has a PXRD pattern that comprises peaks at diffraction angles (2θ) of 8.1±0.1, 18.2±0.1, 18.5±0.1, and 29.8±0.1. Still more particularly, the present invention provides a crystalline form of Compound 1, or a pharmaceutically acceptable salt thereof, that has a PXRD pattern comprising peaks at diffraction angles (2θ) of about 8.1, about 9.1, about 10.6, about 15.4, about 16.3, about 17.4, about 18.2, about 18.5, about 20.0, about 20.8, about 23.2, about 24.0, about 25.9, about 27.4, and about 29.8. Still more particularly, the present invention provides a crystalline form of Compound 1, or a pharmaceutically acceptable salt thereof, that has a PXRD pattern comprising peaks at diffraction angles (2θ) of 8.1±0.1, 9.1±0.1, 10.6±0.1, 15.4±0.1, 16.3±0.1, 17.4±0.1, 18.2±0.1, 18.5±0.1, 20.0±0.1, 20.8±0.1, 23.2±0.1, 24.0±0.1, 25.9±0.1, 27.4±0.1, and 29.8±0.1. Still more particularly, the invention provides a crystalline form of Compound 1, or a pharmaceutically acceptable salt thereof, that has a PXRD pattern comprising peaks at diffraction angles (2θ) essentially the same as shown in FIG. 1A. Even more particularly, the invention provides a crystalline form of Compound 1, or a pharmaceutically acceptable salt thereof, that is characterized by a Differential Scanning Calorimetry (DSC) thermogram essentially the same as shown in FIG. 1B.

In a further embodiment is a pharmaceutical composition that comprises a crystalline form of Compound 1, or a pharmaceutically acceptable salt thereof, that has a PXRD pattern comprising peaks at diffraction angles (2θ) of 8.1±0.1 and 29.8±0.1. Even more particularly, the invention provides a pharmaceutical composition comprising a crystalline form of Compound 1, or a pharmaceutically acceptable salt thereof, that has a PXRD pattern that comprises peaks at diffraction angles (2θ) of 8.1±0.1, 18.2±0.1, 18.5±0.1, and 29.8±0.1. Still more particularly, the present invention provides a pharmaceutical composition comprising a crystalline form of Compound 1, or a pharmaceutically acceptable salt thereof, that has a PXRD pattern comprising peaks at diffraction angles (2θ) of 8.1±0.1, 9.1±0.1, 10.6±0.1, 15.4±0.1, 16.3±0.1, 17.4±0.1, 18.2±0.1, 18.5±0.1, 20.0±0.1, 20.8±0.1, 23.2±0.1, 24.0±0.1, 25.9±0.1, 27.4±0.1, and 29.8±0.1.

In another embodiment are methods for producing polymorphic Form I of Compound 1, comprising preparing a slurry comprising 6-[2-(methylcarbamoyl)phenylsulfanyl]-3-E-[2-(pyridin-2-yl)ethenyl]indazole and an alcohol such as methanol, heating the slurry between about 40° C. to about 60° C., adding water to the slurry, cooling the slurry, and separating the solid portion from the other components of the slurry.

In another embodiment, the invention provides a crystalline form of Compound 1, or a pharmaceutically acceptable salt thereof, wherein the crystalline form is a polymorph designated as Form II. In a further embodiment, the invention provides a crystalline form of Compound 1, or a pharmaceutically acceptable salt thereof, wherein the crystalline form is a substantially pure polymorph of Form II. In a further embodiment, the invention provides a crystalline form of Compound 1, or a pharmaceutically acceptable salt thereof, that has a PXRD pattern comprising peaks at diffraction angles (2θ) of about 8.5 and about 18.8. Even more particularly, the invention provides a crystalline form of Compound 1, or a pharmaceutically acceptable salt thereof, that has a PXRD pattern comprising peaks at diffraction angles (2θ) of 8.5±0.1 and 18.8±0.1. Even more particularly, the invention provides a crystalline form of Compound 1, or a pharmaceutically acceptable salt thereof, that has a PXRD pattern comprising peaks at diffraction angles (2θ) of about 8.5, about 10.9, about 14.8, and about 18.8. Even more particularly, the invention provides a crystalline form of Compound 1, or a pharmaceutically acceptable salt thereof, that has a PXRD pattern that comprises peaks at diffraction angles (26) of 8.5±0.1, 10.9±0.1, 14.8±0.1, and 18.8±0.1. Still more particularly, the present invention provides a crystalline form of Compound 1, or a pharmaceutically acceptable salt thereof, that has a PXRD pattern comprising peaks at diffraction angles (2θ) of about 8.5, about 10.9, about 14.8, about 16.2, about 18.8, about 21.5, about 24.8, about 25.9, about 30.3, and about 32.2. Still more particularly, the present invention provides a crystalline form of Compound 1, or a pharmaceutically acceptable salt thereof, that has a PXRD pattern comprising peaks at diffraction angles (2θ) of 8.5±0.1, 10.9±0.1, 14.8±0.1, 16.2±0.1, 18.8±0.1, 21.5±0.1, 24.8±0.1, 25.9±0.1, 30.3±0.1, and 32.2±0.1. Still more particularly, the invention provides a crystalline form of Compound 1, or a pharmaceutically acceptable salt thereof, that has a PXRD pattern comprising peaks at diffraction angles (2θ) essentially the same as shown in FIG. 2A. Even more particularly, the invention provides a crystalline form of Compound 1, or a pharmaceutically acceptable salt thereof, that is characterized by a Differential Scanning Calorimetry (DSC) thermogram essentially the same as shown in FIG. 2B.

In a further embodiment is a pharmaceutical composition that comprises a crystalline form of Compound 1, or a pharmaceutically acceptable salt thereof, that has a PXRD pattern comprising peaks at diffraction angles (2θ) of 8.5±0.1 and 18.8±0.1. Even more particularly, the invention provides a pharmaceutical composition comprising a crystalline form of Compound 1, or a pharmaceutically acceptable salt thereof, that has a PXRD pattern that comprises peaks at diffraction angles (2θ) of 8.5±0.1, 10.9±0.1, 14.8 ±0.1, and 18.8±0.1. Still more particularly, the present invention provides a pharmaceutical composition comprising a crystalline form of Compound 1, or a pharmaceutically acceptable salt thereof, that has a PXRD pattern comprising peaks at diffraction angles (2θ) of 8.5±0.1, 10.9±0.1, 14.8±0.1, 16.2±0.1, 18.8±0.1, 21.5±0.1, 24.8±0.1, 25.9±0.1, 30.3±0.1, and 32.2±0.1.

In another embodiment are methods for producing polymorphic Form II of Compound 1, comprising exposing 6-[2-(methylcarbamoyl)phenylsulfanyl]-3-E-[2-(pyridin-2-yl)ethenyl]indazole to humidity at ambient temperature. In a further aspect, the humidity is at least a relative humidity of 80%.

In another embodiment, the invention provides a crystalline form of Compound 1, or a pharmaceutically acceptable salt thereof, wherein the crystalline form is a polymorph designated as Form III. In a further embodiment, the invention provides a crystalline form of Compound 1, or a pharmaceutically acceptable salt thereof, wherein the crystalline form is a substantially pure polymorph of Form III. In a further embodiment, the invention provides a crystalline form of Compound 1, or a pharmaceutically acceptable salt thereof, that has a PXRD pattern comprising peaks at diffraction angles (2θ) of about 13.0 and about 24.1. Even more particularly, the invention provides a crystalline form of Compound 1, or a pharmaceutically acceptable salt thereof, that has a PXRD pattern comprising peaks at diffraction angles (2θ) of 13.0±0.1 and 24.1±0.1. Even more particularly, the invention provides a crystalline form of Compound 1, or a pharmaceutically acceptable salt thereof, that has a PXRD pattern comprising peaks at diffraction angles (2θ) of about 13.0, about 13.3, about 21.7, and about 24.1. Even more particularly, the invention provides a crystalline form of Compound 1, or a pharmaceutically acceptable salt thereof, that has a PXRD pattern that comprises peaks at diffraction angles (2θ) of 13.0±0.1, 13.3±0.1, 21.7±0.1, and 24.1±0.1. Still more particularly, the present invention provides a crystalline form of Compound 1, or a pharmaceutically acceptable salt thereof, that has a PXRD pattern comprising peaks at diffraction angles (2θ) of about 10.5, about 13.0, about 13.3, about 15.8, about 16.4, about 17.5, about 19.5, about 20.1, about 21.4, about 21.7, about 24.1, about 25.0, and about 26.9. Still more particularly, the present invention provides a crystalline form of Compound 1, or a pharmaceutically acceptable salt thereof, that has a PXRD pattern comprising peaks at diffraction angles (2θ) of 10.5±0.1, 13.0±0.1, 13.3±0.1, 15.8±0.1, 16.4±0.1, 17.5±0.1, 19.5±0.1, 20.1±0.1, 21.4±0.1, 21.7±0.1, 24.1±0.1, 25.0±0.1, and 26.9±0.1. Still more particularly, the invention provides a crystalline form of Compound 1, or a pharmaceutically acceptable salt thereof, that has a PXRD pattern comprising peaks at diffraction angles (2θ) essentially the same as shown in FIG. 3A. Even more particularly, the invention provides a crystalline form of Compound 1, or a pharmaceutically acceptable salt thereof, that is characterized by a Differential Scanning Calorimetry (DSC) thermogram essentially the same as shown in FIG. 3B.

In a further embodiment is a pharmaceutical composition that comprises a crystalline form of Compound 1, or a pharmaceutically acceptable salt thereof, that has a PXRD pattern comprising peaks at diffraction angles (2θ) of 13.0±0.1 and 24.1±0.1. Even more particularly, the invention provides a pharmaceutical composition comprising a crystalline form of Compound 1, or a pharmaceutically acceptable salt thereof, that has a PXRD pattern that comprises peaks at diffraction angles (2θ) of 13.0±0.1, 13.3±0.1, 21.7±0.1, and 24.1±0.1. Still more particularly, the present invention provides a pharmaceutical composition comprising a crystalline form of Compound 1, or a pharmaceutically acceptable salt thereof, that has a PXRD pattern comprising peaks at diffraction angles (2θ) of 10.5±0.1, 13.0±0.1, 13.3±0.1, 15.8±0.1, 16.4±0.1, 17.5±0.1, 19.5±0.1, 20.1±0.1, 21.4±0.1, 21.7±0.1, 24.1±0.1, 25.0±0.1, and 26.9±0.1.

In another embodiment are methods for producing polymorphic Form III of Compound 1, comprising preparing a slurry comprising a pharmaceutically acceptable salt of 6-[2-(methylcarbamoyl)phenylsulfanyl]-3-E-[2-(pyridin-2-yl)ethenyl]indazole, a base and an aprotic solvent, heating and stirring the slurry to a temperature between about 45° C. and about 80° C., and separating solid portion from the other components of the slurry. In a further aspect the aprotic solvent is ethyl acetate. In yet a further aspect, the base is NaHCO₃.

In another embodiment, the invention provides a crystalline form of Compound 1, or a pharmaceutically acceptable salt thereof, wherein the crystalline form is a polymorph designated as Form IV. In a further embodiment, the invention provides a crystalline form of Compound 1, or a pharmaceutically acceptable salt thereof, wherein the crystalline form is a substantially pure polymorph of Form IV. In a further embodiment, the invention provides a crystalline form of Compound 1, or a pharmaceutically acceptable salt thereof, that has a PXRD pattern comprising peaks at diffraction angles (2θ) of about 8.9 and about 15.7. Even more particularly, the invention provides a crystalline form of Compound 1, or a pharmaceutically acceptable salt thereof, that has a PXRD pattern comprising peaks at diffraction angles (2θ) of 8.9±0.1 and 15.7±0.1. Even more particularly, the invention provides a crystalline form of Compound 1, or a pharmaceutically acceptable salt thereof, that has a PXRD pattern comprising peaks at diffraction angles (2θ) of about 8.9, about 14.6, about 15.7, and about 19.2. Even more particularly, the invention provides a crystalline form of Compound 1, or a pharmaceutically acceptable salt thereof, that has a PXRD pattern that comprises peaks at diffraction angles (2θ) of 8.9±0.1, 14.6±0.1, 15.7±0.1, and 19.2±0.1. Still more particularly, the present invention provides a crystalline form of Compound 1, or a pharmaceutically acceptable salt thereof, that has a PXRD pattern comprising peaks at diffraction angles (2θ) of about 8.9, about 12.0, about 14.6, about 15.2, about 15.7, about 17.8, about 19.2, about 20.5, about 21.6, about 23.2, about 24.2, about 24.8, about 26.2, and about 27.5. Still more particularly, the present invention provides a crystalline form of Compound 1, or a pharmaceutically acceptable salt thereof, that has a PXRD pattern comprising peaks at diffraction angles (2θ) of 8.9±0.1, 12.0±0.1, 14.6±0.1, 15.2±0.1, 15.7±0.1, 17.8±0.1, 19.2±0.1, 20.5±0.1, 21.6±0.1, 23.2±0.1, 24.2±0.1, 24.8±0.1, 26.2±0.1, and 27.5±0.1. Still more particularly, the invention provides a crystalline form of Compound 1, or a pharmaceutically acceptable salt thereof, that has a PXRD pattern comprising peaks at diffraction angles (2θ) essentially the same as shown in FIG. 4A. Even more particularly, the invention provides a crystalline form of Compound 1, or a pharmaceutically acceptable salt thereof, that is characterized by a Differential Scanning Calorimetry (DSC) thermogram essentially the same as shown in FIG. 4B.

In a further embodiment is a pharmaceutical composition that comprises a crystalline form of Compound 1, or a pharmaceutically acceptable salt thereof, that has a PXRD pattern comprising peaks at diffraction angles (2θ) of 8.9±0.1 and 15.7±0.1. Even more particularly, the invention provides a pharmaceutical composition comprising a crystalline form of Compound 1, or a pharmaceutically acceptable salt thereof, that has a PXRD pattern that comprises peaks at diffraction angles (2θ) of 8.9±0.1, 14.6±0.1, 15.7 ±0.1, and 19.2±0.1. Still more particularly, the present invention provides a pharmaceutical composition comprising a crystalline form of Compound 1, or a pharmaceutically acceptable salt thereof, that has a PXRD pattern comprising peaks at diffraction angles (2θ) of 8.9±0.1, 12.0±0.1, 14.6±0.1, 15.2±0.1, 15.7±0.1, 17.8±0.1, 19.2±0.1, 20.5±0.1, 21.6±0.1, 23.2±0.1, 24.2±0.1, 24.8±0.1, 26.2±0.1, and 27.5±0.1.

In another embodiment are methods for producing polymorphic Form IV of Compound 1 from a different polymorphic form of 6-[2-(methylcarbamoyl)phenylsulfanyl]-3-E-[2-(pyridin-2-yl)ethenyl]indazole, comprising heating the different polymorphic form, wherein the different polymorphic form is hydrated or solvated. In a further aspect, the heating occurs under vacuum. In yet a further aspect the heating is conducted between about 110° C. and about 135° C. And in yet a further aspect, the solvate of the different polymorphic form is selected from the group consisting of a solvate of methanol, a solvate of ethanol, and a solvate of ethyl acetate. In yet a further aspect the different polymorphic form is polymorphic Form III of Compound 1.

In a further aspect of this embodiment are methods for converting polymorphic Form VI of Compound 1 into polymorphic Form IV of Compound 1 comprising heating a slurry of polymorphic Form VI of Compound 1 in an aromatic solvent, and isolating the solid portion from the other components of the slurry. In a further aspect, the heating step occurs at a temperature of at least 110° C.

In a further aspect of this embodiment are methods for producing polymorphic Form IV of Compound 1, comprising heating a slurry comprising a hydrated form of Compound 1 and an aromatic solvent between about 110 and about 140° C., and separating the solid portion from the other components of the slurry. In yet a further aspect, the aromatic solvent is toluene or xylenes. In yet a further aspect, the hydrated form of Compound 1 is the polymorphic Form III of Compound 1.

In a further aspect of this embodiment are methods for producing polymorphic Form IV of Compound 1, comprising recrystallizing Compound 1 to form a recrystallized product, heating a slurry comprising the recrystallized product and an aromatic solvent between about 110° C. and about 150° C., and separating the solid portion from the other components of the slurry. In yet a further aspect, Compound 1 is recrystallized from a solution comprising dichloromethane and methanol. In yet a further aspect, the aromatic solvent is toluene or xylenes.

In a further aspect of this embodiment are methods for producing polymorphic Form IV of Compound 1, comprising recrystallizing 6-[2-(methylcarbamoyl)phenylsulfanyl]-3-E-[2-(pyridin-2-yl)ethenyl]indazole from a solution of a water soluble polymer, adding water to the solution to precipitate solids, and separating precipitated solids from the water soluble polymer and water. In yet a further aspect, the water soluble polymer is (poly)ethyleneglycol. In still a further aspect, the (poly)ethyleneglycol is PEG-400.

In another embodiment, the invention provides a crystalline form of Compound 1, or a pharmaceutically acceptable salt thereof, wherein the crystalline form is a polymorph designated as Form VI. In a further embodiment, the invention provides a crystalline form of Compound 1, or a pharmaceutically acceptable salt thereof, wherein the crystalline form is a substantially pure polymorph of Form VI. In a further embodiment, the invention provides a crystalline form of Compound 1, or a pharmaceutically acceptable salt thereof, that has a PXRD pattern comprising peaks at diffraction angles (2θ) of about 9.6 and about 18.1. Even more particularly, the invention provides a crystalline form of Compound 1, or a pharmaceutically acceptable salt thereof, that has a PXRD pattern comprising peaks at diffraction angles (2θ) of 9.6±0.1 and 18.1±0.1. Even more particularly, the invention provides a crystalline form of Compound 1, or a pharmaceutically acceptable salt thereof, that has a PXRD pattern comprising peaks at diffraction angles (2θ) of about 9.6, about 11.6, about 18.1, and about 25.2. Even more particularly, the invention provides a crystalline form of Compound 1, or a pharmaceutically acceptable salt thereof, that has a PXRD pattern that comprises peaks at diffraction angles (2θ) of 9.6±0.1, 11.6±0.1, 18.1±0.1, and 25.2±0.1. Still more particularly, the present invention provides a crystalline form of Compound 1, or a pharmaceutically acceptable salt thereof, that has a PXRD pattern comprising peaks at diffraction angles (2θ) of about 9.6, about 11.6, about 17.5, about 18.1, about 19.9, and about 25.2. Still more particularly, the present invention provides a crystalline form of Compound 1, or a pharmaceutically acceptable salt thereof, that has a PXRD pattern comprising peaks at diffraction angles (2θ) of 9.6±0.1, 11.6±0.1, 17.5±0.1, 18.1±0.1, 19.9±0.1, and 25.2±0.1. Still more particularly, the invention provides a crystalline form of Compound 1, or a pharmaceutically acceptable salt thereof, that has a PXRD pattern comprising peaks at diffraction angles (2θ) essentially the same as shown in FIG. 5A. Even more particularly, the invention provides a crystalline form of Compound 1, or a pharmaceutically acceptable salt thereof, that is characterized by a Differential Scanning Calorimetry (DSC) thermogram essentially the same as shown in FIG. 5B.

In a further embodiment is a pharmaceutical composition that comprises a crystalline form of Compound 1, or a pharmaceutically acceptable salt thereof, that has a PXRD pattern comprising peaks at diffraction angles (2θ) of 9.6±0.1 and 18.1±0.1. Even more particularly, the invention provides a pharmaceutical composition comprising a crystalline form of Compound 1, or a pharmaceutically acceptable salt thereof, that has a PXRD pattern that comprises peaks at diffraction angles (2θ) of 9.6±0.1, 11.6±0.1, 18.1 ±0.1, and 25.2±0.1. Still more particularly, the present invention provides a pharmaceutical composition comprising a crystalline form of Compound 1, or a pharmaceutically acceptable salt thereof, that has a PXRD pattern comprising peaks at diffraction angles (2θ) of 9.6±0.1, 11.6±0.1, 17.5±0.1, 18.1±0.1, 19.9±0.1, and 25.2±0.1.

In another embodiment are methods for producing polymorphic Form VI of Compound 1 comprising preparing a slurry comprising a pharmaceutically acceptable salt of 6-[2-(methylcarbamoyl)phenylsulfanyl]-3-E-[2-(pyridin-2-yl)ethenyl]indazole, a base and a protic solvent, heating and stirring the slurry between about 45° C. and about 80° C., and separating the solid portion from the other components of the slurry. In yet a further aspect, the protic solvent is an alcohol. In yet a further aspect, the protic solvent is ethanol. In still a further aspect, the base is NaHCO₃.

In another embodiment, the invention provides a crystalline form of Compound 1, or a pharmaceutically acceptable salt thereof, wherein the crystalline form is a polymorph designated as Form VII. In a further embodiment, the invention provides a crystalline form of Compound 1, or a pharmaceutically acceptable salt thereof, wherein the crystalline form is a substantially pure polymorph of Form VII. In a further embodiment, the invention provides a crystalline form of Compound 1, or a pharmaceutically acceptable salt thereof, that has a PXRD pattern comprising peaks at diffraction angles (2θ) of about 9.4 and about 17.0. Even more particularly, the invention provides a crystalline form of Compound 1, or a pharmaceutically acceptable salt thereof, that has a PXRD pattern comprising peaks at diffraction angles (2θ) of 9.4±0.1 and 17.0±0.1. Even more particularly, the invention provides a crystalline form of Compound 1, or a pharmaceutically acceptable salt thereof, that has a PXRD pattern comprising peaks at diffraction angles (2θ) of about 9.4, about 17.0, about 23.6, and about 25.1. Even more particularly, the invention provides a crystalline form of Compound 1, or a pharmaceutically acceptable salt thereof, that has a PXRD pattern that comprises peaks at diffraction angles (2θ) of 9.4±0.1, 17.0±0.1, 23.6±0.1, and 25.1±0.1. Still more particularly, the present invention provides a crystalline form of Compound 1, or a pharmaceutically acceptable salt thereof, that has a PXRD pattern comprising peaks at diffraction angles (2θ) of about 9.4, about 10.2, about 16.2, about 17.0, about 18.9, about 19.7, about 21.5, about 22.7, about 23.6, about 25.1, about 26.2, about 27.4, and about 29.3. Still more particularly, the present invention provides a crystalline form of Compound 1, or a pharmaceutically acceptable salt thereof, that has a PXRD pattern comprising peaks at diffraction angles (2θ) of 9.4±0.1, 10.2±0.1, 16.2±0.1, 17.0±0.1, 18.9±0.1, 19.7±0.1, 21.5±0.1, 22.7±0.1, 23.6±0.1, 25.1±0.1, 26.2±0.1, 27.4±0.1, and 29.3±0.1. Still more particularly, the invention provides a crystalline form of Compound 1, or a pharmaceutically acceptable salt thereof, that has a PXRD pattern comprising peaks at diffraction angles (2θ) essentially the same as shown in FIG. 6A. Even more particularly, the invention provides a crystalline form of Compound 1, or a pharmaceutically acceptable salt thereof, that is characterized by a Differential Scanning Calorimetry (DSC) thermogram essentially the same as shown in FIG. 6B.

In a further embodiment is a pharmaceutical composition that comprises a crystalline form of Compound 1, or a pharmaceutically acceptable salt thereof, that has a PXRD pattern comprising peaks at diffraction angles (2θ) of 9.4±0.1 and 17.0±0.1. Even more particularly, the invention provides a pharmaceutical composition comprising a crystalline form of Compound 1, or a pharmaceutically acceptable salt thereof, that has a PXRD pattern that comprises peaks at diffraction angles (2θ) of 9.4±0.1, 17.0±0.1, 23.6±0.1, and 25.1±0.1. Still more particularly, the present invention provides a pharmaceutical composition comprising a crystalline form of Compound 1, or a pharmaceutically acceptable salt thereof, that has a PXRD pattern comprising peaks at diffraction angles (2θ) of 9.4±0.1, 10.2±0.1, 16.2±0.1, 17.0±0.1, 18.9±0.1, 19.7±0.1, 21.5±0.1, 22.7±0.1, 23.6±0.1, 25.1±0.1, 26.2±0.1, 27.4±0.1, and 29.3±0.1.

In another embodiment are methods for producing polymorphic Form VII of Compound 1 comprising preparing a slurry comprising 6-[2-(methylcarbamoyl)phenylsulfanyl]-3-E-[2-(pyridin-2-yl)ethenyl]indazole or a solvate thereof and a protic solvent; heating and stirring the slurry between about 45° C. and about 80° C.; and separating the solid portion from the other components of the slurry. In yet a further aspect, the protic solvent is isopropanol.

In another embodiment, the invention provides a crystalline form of Compound 1, or a pharmaceutically acceptable salt thereof, wherein the crystalline form is a polymorph designated as Form VIII. In a further embodiment, the invention provides a crystalline form of Compound 1, or a pharmaceutically acceptable salt thereof, wherein the crystalline form is a substantially pure polymorph of Form VIII. In a further embodiment, the invention provides a crystalline form of Compound 1, or a pharmaceutically acceptable salt thereof, that has a PXRD pattern comprising peaks at diffraction angles (2θ) of about 24.6 and about 26.3. Even more particularly, the invention provides a crystalline form of Compound 1, or a pharmaceutically acceptable salt thereof, that has a PXRD pattern comprising peaks at diffraction angles (2θ) of 24.6±0.1 and 26.3±0.1. Even more particularly, the invention provides a crystalline form of Compound 1, or a pharmaceutically acceptable salt thereof, that has a PXRD pattern comprising peaks at diffraction angles (2θ) of about 24.6, about 25.9, about 26.3, and about 32.0. Even more particularly, the invention provides a crystalline form of Compound 1, or a pharmaceutically acceptable salt thereof, that has a PXRD pattern that comprises peaks at diffraction angles (2θ) of 24.6±0.1, 25.9±0.1, 26.3 ±0.1, and 32.0±0.1. Still more particularly, the present invention provides a crystalline form of Compound 1, or a pharmaceutically acceptable salt thereof, that has a PXRD pattern comprising peaks at diffraction angles (2θ) of about 10.7, about 15.5, about 15.9, about 20.6, about 22.7, about 24.6, about 25.9, about 26.3, and about 32.0. Still more particularly, the present invention provides a crystalline form of Compound 1, or a pharmaceutically acceptable salt thereof, that has a PXRD pattern comprising peaks at diffraction angles (2θ) of 10.7±0.1, 15.5±0.1, 15.9±0.1, 20.6±0.1, 22.7±0.1, 24.6±0.1, 25.9±0.1, 26.3±0.1, and 32.0±0.1. Still more particularly, the invention provides a crystalline form of Compound 1, or a pharmaceutically acceptable salt thereof, that has a PXRD pattern comprising peaks at diffraction angles (2θ) essentially the same as shown in FIG. 7.

In a further embodiment is a pharmaceutical composition that comprises a crystalline form of Compound 1, or a pharmaceutically acceptable salt thereof, that has a PXRD pattern comprising peaks at diffraction angles (26) of 24.6±0.1 and 26.3±0.1. Even more particularly, the invention provides a pharmaceutical composition comprising a crystalline form of Compound 1, or a pharmaceutically acceptable salt thereof, that has a PXRD pattern that comprises peaks at diffraction angles (2θ) of 24.6±0.1, 25.9±0.1, 26.3±0.1, and 32.0±0.1. Still more particularly, the present invention provides a pharmaceutical composition comprising a crystalline form of Compound 1, or a pharmaceutically acceptable salt thereof, that has a PXRD pattern comprising peaks at diffraction angles (2θ) of 10.7±0.1, 15.5±0.1, 15.9±0.1, 20.6±0.1, 22.7±0.1, 24.6±0.1, 25.9±0.1, 26.3±0.1, and 32.0±0.1.

In another embodiment are methods for producing polymorphic Form VIII of Compound 1 comprising dissolving 6-[2-(methylcarbamoyl)phenylsulfanyl]-3-E-[2-(pyridin-2-yl)ethenyl]indazole in a minimal amount of refluxing aprotic solvent forming a solution; cooling the solution, whereupon crystals form; and isolating crystalline product. In yet a further aspect, the aprotic solvent is dioxane.

In another embodiment of the present invention is a solid form of Compound 1, or a pharmaceutically acceptable salt thereof, wherein the solid form comprises at least two of the following crystalline forms: polymorph Forms I, II, III, IV, VI, VII, and VIII.

In yet a further aspect of the present invention are pharmaceutical compositions comprising the polymorphic Form I of Compound 1. In a further aspect are methods of treating a mammalian disease condition mediated by protein kinase activity comprising administering a therapeutically effective amount of polymorphic Form I of Compound 1. In yet a further aspect are methods of treating a hyperproliferative disorder in a mammal, such as tumor growth, cell proliferation, or angiogenesis, comprising administering a therapeutically effective amount of polymorphic Form I of Compound 1. In a further aspect are methods of treating a mammalian disease condition mediated by VEGF activity, comprising administering to a mammal in need thereof a therapeutically effective amount of polymorphic Form I of Compound 1.

In yet a further aspect of the present invention are pharmaceutical compositions comprising the polymorphic Form II of Compound 1. In a further aspect are methods of treating a mammalian disease condition mediated by protein kinase activity comprising administering a therapeutically effective amount of polymorphic Form II of Compound 1. In yet a further aspect are methods of treating a hyperproliferative disorder in a mammal, such as tumor growth, cell proliferation, or angiogenesis, comprising administering a therapeutically effective amount of polymorphic Form II of Compound 1. In a further aspect are methods of treating a mammalian disease condition mediated by VEGF activity, comprising administering to a mammal in need thereof a therapeutically effective amount of polymorphic Form II of Compound 1.

In yet a further aspect of the present invention are pharmaceutical compositions comprising the polymorphic Form III of Compound 1. In a further aspect are methods of treating a mammalian disease condition mediated by protein kinase activity comprising administering a therapeutically effective amount of polymorphic Form III of Compound 1. In yet a further aspect are methods of treating a hyperproliferative disorder in a mammal, such as tumor growth, cell proliferation, or angiogenesis, comprising administering a therapeutically effective amount of polymorphic Form III of Compound 1. In a further aspect are methods of treating a mammalian disease condition mediated by VEGF activity, comprising administering to a mammal in need thereof a therapeutically effective amount of polymorphic Form III of Compound 1.

In yet a further aspect of the present invention are pharmaceutical compositions comprising the polymorphic Form IV of Compound 1. In a further aspect are methods of treating a mammalian disease condition mediated by protein kinase activity comprising administering a therapeutically effective amount of polymorphic Form IV of Compound 1. In yet a further aspect are methods of treating a hyperproliferative disorder in a mammal, such as tumor growth, cell proliferation, or angiogenesis, comprising administering a therapeutically effective amount of polymorphic Form IV of Compound 1. In a further aspect are methods of treating a mammalian disease condition mediated by VEGF activity, comprising administering to a mammal in need thereof a therapeutically effective amount of polymorphic Form IV of Compound 1.

In yet a further aspect of the present invention are pharmaceutical compositions comprising the polymorphic Form VI of Compound 1. In a further aspect are methods of treating a mammalian disease condition mediated by protein kinase activity comprising administering a therapeutically effective amount of polymorphic Form VI of Compound 1. In yet a further aspect are methods of treating a hyperproliferative disorder in a mammal, such as tumor growth, cell proliferation, or angiogenesis, comprising administering a therapeutically effective amount of polymorphic Form VI of Compound 1. In a further aspect are methods of treating a mammalian disease condition mediated by VEGF activity, comprising administering to a mammal in need thereof a therapeutically effective amount of polymorphic Form VI of Compound 1.

In yet a further aspect of the present invention are pharmaceutical compositions comprising the polymorphic Form VII of Compound 1. In a further aspect are methods of treating a mammalian disease condition mediated by protein kinase activity comprising administering a therapeutically effective amount of polymorphic Form VII of Compound 1. In yet a further aspect are methods of treating a hyperproliferative disorder in a mammal, such as tumor growth, cell proliferation, or angiogenesis, comprising administering a therapeutically effective amount of polymorphic Form VII of Compound 1. In a further aspect are methods of treating a mammalian disease condition mediated by VEGF activity, comprising administering to a mammal in need thereof a therapeutically effective amount of polymorphic Form VII of Compound 1.

In yet a further aspect of the present invention are pharmaceutical compositions comprising the polymorphic Form VIII of Compound 1. In a further aspect are methods of treating a mammalian disease condition mediated by protein kinase activity comprising administering a therapeutically effective amount of polymorphic Form VIII of Compound 1. In yet a further aspect are methods of treating a hyperproliferative disorder in a mammal, such as tumor growth, cell proliferation, or angiogenesis, comprising administering a therapeutically effective amount of polymorphic Form VIII of Compound 1. In a further aspect are methods of treating a mammalian disease condition mediated by VEGF activity, comprising administering to a mammal in need thereof a therapeutically effective amount of polymorphic Form VIII of Compound 1.

The present invention is further directed to methods of modulating or inhibiting protein kinase activity (e.g., receptors for VEGF, VEGF, FGF, CDK complexes, TEK, CHK1, LCK, FAK, and phosphorylase kinase among others), for example in mammalian tissue, by administering at least one polymorphic form of Compound 1.

The present invention is also directed to combination therapeutic methods of treating a hyperproliferative disorder, or a disease condition mediated by VEGF activity, which comprises administering to a mammal in need thereof a therapeutically effective amount of a pharmaceutical composition which comprises any of the polymorphic forms, or pharmaceutical compositions discussed above, in combination with a therapeutically effective amount of one or more substances selected from anti-tumor agents, anti-angiogenesis agents, signal transduction inhibitors, and antiproliferative agents.

The term “active agent” or “active ingredient” refers to a polymorphic form of Compound 1, or to a solid form that comprises two or more polymorphic forms of Compound 1.

The term “ambient temperature” refers to a temperature condition typically encountered in a laboratory setting. This includes the approximate temperature range of about 20 to about 30° C.

The term “aqueous base” refers to any organic or inorganic base. Aqueous bases include, by way of example only, metal bicarbonates, such as sodium bicarbonate, potassium carbonate, cesium carbonate, and the like.

The term “aromatic solvent” refers to an organic solvent possessing an aromatic moiety, including by way of example only, benzene, toluene, xylene isomers or mixtures thereof, and the like.

The term “chemical stability” refers to a type of stability in which a particular compound maintains its chemical integrity, and includes, but is not limited to, thermal stability, light stability, and moisture stability.

The term ‘detectable amount’ refers to an amount or amount per unit volume that can be detected using conventional techniques, such as X-ray powder diffraction, differential scanning calorimetry, HPLC, FT-IR, Raman spectroscopy, and the like.

The term “exposing to humidity” refers to the process of exposing a substance to water vapor in a humidor, humidity chamber, or any apparatus capable of controlling relative humidity. The term may also describe the process of exposing a substance to ambient humidity as during storage.

The term ‘hyperproliferative disorder’ refers to abnormal cell growth that is independent of normal regulatory mechanisms (e.g., loss of contact inhibition), including the abnormal growth of normal cells and the growth of abnormal cells. This includes, but is not limited to, the abnormal growth of tumor cells (tumors), both benign and malignant. Examples of such benign proliferative diseases are psoriasis, benign prostatic hypertrophy, human papilloma virus (HPV), and restinosis. The term “hyperproliferative disorder” also refers to cancer, including, but not limited to, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. In another embodiment of said method, said abnormal cell growth is a benign proliferative disease, including, but not limited to, psoriasis, benign prostatic hypertrophy or restinosis.

The term “inert solvent” refers to any solvent or liquid component of a slurry that does not chemically react with other components in a solution or slurry. Inert solvents include, by way of example only aprotic solvents such as aromatic solvents, ethyl acetate, acetone, methyl tert-butylether, dioxane, THF, and the like. Protic solvents include, by way of example only, methanol, ethanol, propanol isomers, butanol isomers and the like.

The term “mediated by VEGF activity” refers to biological or molecular processes that are regulated, modulated, or inhibited by VEGF protein kinase activity. For certain applications, inhibition of the protein kinase activity associated with CDK complexes, among others, and those which inhibit angiogenesis and/or inflammation are preferred. The present invention includes methods of modulating or inhibiting protein kinase activity, for example in mammalian tissue, by administering polymorphic forms of Compound 1. The activity of agents as anti-proliferatives is easily measured by known methods, for example by using whole cell cultures in an MTT assay. The activity of polymorphic forms of Compound 1 as mediators of protein kinase activity, such as the activity of kinases, may be measured by any of the methods available to those skilled in the art, including in vivo and/or in vitro assays.

The term ‘minimal amount’ refers to the least amount of solvent required to completely dissolve a substance at a given temperature.

The term “pharmaceutically acceptable salt” refers to a salt that retains the biological effectiveness of the free acids and bases of the specified compound and that is not biologically or otherwise undesirable. A compound of the invention may possess a sufficiently acidic, a sufficiently basic, or both functional groups, and accordingly react with any of a number of inorganic or organic bases, and inorganic and organic acids, to form pharmaceutically acceptable salts. Exemplary pharmaceutically acceptable salts include those salts prepared by reaction of the compounds of the present invention with a mineral or organic acid or an inorganic base, such as salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, para-toluene sulfonates (tosylates), formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycollates, tartrates, methane-sulfonates, propanesulfonates, naphthalene-1-sulfonates, naphthalene-2-sulfonates, and mandelates.

If the inventive compound is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the inventive compound is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

The term “polymorph” refers to different crystalline forms of the same compound and includes, but is not limited to, other solid state molecular forms including hydrates (e.g., bound water present in the crystalline structure) and solvates (e.g., bound solvents other than water) of the same compound.

The term “peak intensities” refers to relative signal intensities within a given X-ray diffraction pattern. Factors which can affect the relative peak intensities are sample thickness and preferred orientation (i.e. the crystalline particles are not distributed randomly).

The term “peak positions” as used herein refers to X-ray reflection positions as measured and observed in X-ray powder diffraction experiments. Peak positions are directly related to the dimensions of the unit cell. The peaks, identified by their respective peak positions, have been extracted from the diffraction patterns for the various polymorphic Forms I, II, III, IV, VI, VII, and VIII of Compound 1.

The term “PEG” refers to poly(ethylene glycol). PEG is commercially available having different ranges of polymer chain lengths and thus viscosities. PEG 400 is soluble in alcohols, acetone, benzene, chloroform, acetic acid, CCl₄, and water.

The term “pharmaceutically acceptable, carrier, diluent, or vehicle” refers to a material (or materials) that may be included with a particular pharmaceutical agent to form a pharmaceutical composition, and may be solid or liquid. Exemplary of solid carriers are lactose, sucrose, talc, gelatin, agar, pectin, acacia, magnesium stearate, stearic acid and the like. Exemplary of liquid carriers are syrup, peanut oil, olive oil, water and the like. Similarly, the carrier or diluent may include time-delay or time-release material known in the art, such as glyceryl monostearate or glyceryl distearate alone or with a wax, ethylcellulose, hydroxypropylmethylcellulose, methylmethacrylate and the like.

The term “pharmaceutical composition” refers to a mixture of one or more of the compounds or polymorphs described herein, or physiologically/pharmaceutically acceptable salts or solvates thereof, with other chemical components, such as physiologically/pharmaceutically acceptable carriers and excipients. The purpose of a pharmaceutical composition is to facilitate administration of a compound to an organism.

The term “recrystallize” refers to the process of completely dissolving a solid in a first solvent with heating if necessary, and then inducing precipitation, usually by cooling the solution, or by adding a second solvent in which the solid is poorly soluble.

The term “relative humidity” refers to the ratio of the amount of water vapor in air at a given temperature to the maximum amount of water vapor that can be held at that temperature and pressure, expressed as a percentage.

The term “relative intensity” refers to an intensity value derived from a sample X-ray diffraction pattern. The complete ordinate range scale for a diffraction pattern is assigned a value of 100. A peak having intensity falling between about 50% to about 100% on this scale intensity is termed very strong (vs); a peak having intensity falling between about 50% to about 25% is termed strong (s). Additional weaker peaks are present in typical diffraction patterns and are also characteristic of a given polymorph.

The term ‘slurry’ refers to a solid substance suspended in a liquid medium, typically water or an organic solvent.

The term ‘separating from’ refers to a step in a synthesis in which the desired agent is isolated from other non-desired agents, including, but not limited to any of the following steps: filtering, washing with extra solvent or water, drying with heat and or under vacuum.

The term “substantially pure” with reference to particular polymorphic forms of Compound 1 means the polymorphic form includes less than 10%, preferably less than 5%, preferably less than 3%, preferably less than 1% by weight of impurities, including other polymorphic forms of Compound 1. Such purity may be determined, for example, by X-ray powder diffraction.

An “effective amount” is intended to mean that amount of an agent that significantly inhibits proliferation and/or prevents de-differentiation of a eukaryotic cell, e.g., a mammalian, insect, plant or fungal cell, and is effective for the indicated utility, e.g., specific therapeutic treatment.

The term “therapeutically effective amount” refers to that amount of the compound or polymorph being administered which will relieve to some extent one or more of the symptoms of the disorder being treated. In reference to the treatment of cancer, a therapeutically effective amount refers to that amount which has at least one of the following effects:
(1) reducing the size of the tumor;
(2) inhibiting (that is, slowing to some extent, preferably stopping) tumor metastasis;
(3) inhibiting to some extent (that is, slowing to some extent, preferably stopping) tumor growth, and
(4) relieving to some extent (or, preferably, eliminating) one or more symptoms associated with the cancer.

The term “2 theta value” or “2θ” refers to the peak position based on the experimental setup of the X-ray diffraction experiment and is a common abscissa unit in diffraction patterns. The experimental setup requires that if a reflection is diffracted when the incoming beam forms an angle theta (θ) with a certain lattice plane, the reflected beam is recorded at an angle 2 theta (2θ).

The terms “treat”, “treating” and “treatment” refer to a method of alleviating or abrogating a hyperproliferative disorder and/or its attendant symptoms. With regard particularly to cancer, these terms simply mean that the life expectancy of an individual affected with a cancer will be increased or that one or more of the symptoms of the disease will be reduced.

The term “under vacuum” refers to typical pressures obtainable by a laboratory oil or oil-free diaphragm vacuum pump.

The term “X-ray powder diffraction pattern” refers to the experimentally observed diffractogram or parameters derived therefrom. X-Ray powder diffraction patterns are characterized by peak position (abscissa) and peak intensities (ordinate).

The term “xylenes” refers to any of the xylene isomers or a mixture thereof.

### BRIEF DESCRIPTION OF THE DRAWINGS

FIG. 1A is an X-ray powder diffraction diagram of polymorphic Form I of Compound 1.

FIG. 1B is a differential scanning calorimetry (DSC) profile of polymorphic Form I of Compound 1. A typical profile displays an endotherm with onset at 183-190° C. at a scan rate of 10° C./min.

FIG. 2A is an X-ray powder diffraction diagram of polymorphic Form II of Compound 1.

FIG. 2B is a differential scanning calorimetry (DSC) profile of polymorphic Form II of Compound 1. A typical profile displays endotherms with onset at 102, 152, and 202° C., followed by an exotherm at 206° C. and another exotherm at 210° C. at a scan rate of 10° C./min.

FIG. 3A is an X-ray powder diffraction diagram of polymorphic Form III of Compound 1.

FIG. 3B is a differential scanning calorimetry (DSC) profile of polymorphic Form III of Compound 1. A typical profile displays endotherms with onset at 125-129° C., followed by another endotherm at 210° C. at a scan rate of 10° C./min.

FIG. 3C is Thermal Gravimetric Analysis (TGA) profile of polymorphic Form III. Desolvation is indicated by 10% sample weight loss at 125-129° C. at a scan rate of 10° C./min.

FIG. 4A is an X-ray powder diffraction diagram of polymorphic Form IV of Compound 1.

FIG. 4B is a differential scanning calorimetry (DSC) profile of polymorphic Form IV of Compound 1. A typical profile displays an endotherm with onset at 216° C. at a scan rate of 10° C./min.

FIG. 5A is an X-ray powder diffraction diagram of polymorphic Form VI of Compound 1.

FIG. 5B is a differential scanning calorimetry (DSC) profile of polymorphic Form VI of Compound 1. A typical profile displays endotherms with onset at about 197° C. and at about 209° C. at a scan rate of 10° C./min.

FIG. 6A is an X-ray powder diffraction diagram of polymorphic Form VII of Compound 1.

FIG. 6B is a differential scanning calorimetry (DSC) profile of polymorphic Form VII of Compound 1. Typical profiles are sample-dependent. A typical sample isolated from refluxing THF has an endotherm at 105° C. followed by an exotherm at 115° C., and then endotherms at 137 and 175° C., at a scan rate of 10° C./min.

FIG. 7 is an X-ray powder diffraction diagram of polymorphic Form VIII of Compound 1.

FIG. 8 is a schematic drawing showing the structures of several human metabolites of Compound 1.

### DETAILED DESCRIPTION OF THE INVENTION

It has surprisingly been found that the substance Compound 1 can exist in more than one polymorphic crystalline form. These forms may be used in a formulated product for the treatment of hyperproliferative indications, including cancer. Each form may have advantage over the others in bioavailability, stability, or manufacturability. Crystalline polymorphic forms of Compound 1 have been discovered which are likely to be more suitable for bulk preparation and handling than other polymorphic forms. Processes for producing these polymorphic forms in high purity are described herein. Another object of the present invention is to provide a process for the preparation of each polymorphic form of Compound 1, substantially free from other polymorphic forms of Compound 1. Additionally it is an object of the present invention to provide pharmaceutical formulations comprising Compound 1 in different polymorphic forms as discussed above, and methods of treating hyperproliferative conditions by administering such pharmaceutical formulations.

I. Polymorphic Forms of Compound 1

Each crystalline form of Compound 1 can be characterized by one or more of the following: X-ray powder diffraction pattern (i.e., X-ray diffraction peaks at various diffraction angles (2θ)), melting point onset (and onset of dehydration for hydrated forms) as illustrated by endotherms of a Differential Scanning Calorimetry (DSC) thermogram, Raman spectral diagram pattern, aqueous solubility, light stability under International Conference on Harmonization (ICH) high intensity light conditions, and physical and chemical storage stability. For example, samples of polymorphic Forms I, II, III, IV, VI, VII, and VIII of Compound 1 were each characterized by the positions and relative intensities of peaks in their X-ray powder diffraction patterns. The X-ray powder diffraction parameters differ for each of the polymorphic Forms I, II, III, IV, VI, VII, and VIII of Compound 1. These polymorphic forms of Compound 1 can therefore be distinguished using X-ray powder diffraction.

The X-ray powder diffraction pattern for each polymorph or amorphous form of Compound 1 was measured on a Shimadzu XRD-6000 X-ray diffractometer equipped with a Cu Kα X-ray radiation source (1.5406 Å) operated at 40 kV and 50 mA. Samples were placed in a sample holder and then packed and smoothed with a glass slide. During analysis, the samples were rotated at 60 rpm and analyzed from angles of 4 to 40 degrees (θ-2θ) at 5 degrees per minute with a 0.04 degree step or at 2 degrees per minute with a 0.02 degree step. If limited material was available, samples were placed on a silicon plate (zero background) and analyzed without rotation. The X-Ray diffraction peaks, characterized by peak positions and intensity assignments, have been extracted from the X-ray powder diffractogram of each of the polymorphic forms of Compound 1. One of skill in the art will appreciate that the peak positions (2θ) will show some inter-apparatus variability, typically as much as 0.1 degrees. Accordingly, where peak positions (2θ) are reported, one of skill in the art will recognize that such numbers are intended to encompass such inter-apparatus variability. Furthermore, where the crystalline forms of the present invention are described as having a powder X-ray diffraction pattern essentially the same as that shown in a given figure, the term “essentially the same” is also intended to encompass such inter-apparatus variability in diffraction peak positions. Further, one skilled in the art will appreciate that relative peak intensities will show inter-apparatus variability as well as variability due to degree of crystallinity, preferred orientation, prepared sample surface, and other factors known to those skilled in the art, and should be taken as qualitative measures only.

Different polymorphic forms of Compound 1 were also distinguished using differential scanning calorimetry (DSC). DSC measures the difference in heat energy uptake between a sample solution and an appropriate reference solvent with increase in temperature. DSC thermograms are characterized by endotherms (indicating energy uptake) and also by exotherms (indicating energy release), typically as the sample is heated. The DSC thermographs were obtained using a Mettler Toledo DSC821 instrument at a scan rate of 10° C./min over a temperature range of 30-250° C. Samples were weighed into 40 μl aluminum crucibles that were sealed and punctured with a single hole. The extrapolated onset of melting temperature and, where applicable, the onset of dehydration temperature, were also calculated. Depending upon the rate of heating (i.e., the scan rate) at which the DSC analysis is conducted, the way the DSC on-set temperature is defined and determined, the calibration standard used, the instrument calibration, and the relative humidity and chemical purity of the sample, the endotherms exhibited by the compounds of the invention may vary (by about 0.01-5° C., for crystal polymorph melting and by about 0.01-20° C. for polymorph dehydration) above or below the endotherms. For any given example, the observed endotherms may also differ from instrument to instrument; however, it will generally be within the ranges defined herein provided the instruments are calibrated similarly.

Different polymorphic forms of Compound 1 were also distinguished using thermal gravimetric analysis (TGA). TGAs were performed on a Mettler Toledo TGA 500 instrument. TGA is a testing procedure in which changes in weight of a specimen are recorded as the specimen is heated in air or in a controlled atmosphere such as nitrogen. Thermogravimetric curves (thermograms) provide information regarding solvent and water content and the thermal stability of materials.

Different polymorphic forms of Compound 1, may also be distinguished by different stabilities and different solubilities.

In one embodiment, the polymorphic forms of the present invention are substantially pure, meaning each polymorphic form of Compound 1 includes less than 10%, for example less than 5%, or for example less than 3%, or even further, for example, less than 1% by weight of impurities, including other polymorphic forms of Compound 1.

The solid forms of the present invention may also comprise more than one polymorphic form. One of skill in the art will recognize that crystalline forms of a given compound can exist in substantially pure forms of a single polymorph, and can also exist in a crystalline form that comprises two or more different polymorphs. Where a solid form comprises two or more polymorphs, the X-ray diffraction pattern will have peaks characteristic of each of the individual polymorphs of the present invention. For example, a solid form that comprises two polymorphs will have a powder X-ray diffraction pattern that is a convolution of the two X-ray diffraction patterns that correspond to the substantially pure polymorphic forms. In one embodiment, for example, a solid form of the present invention containing a first and second polymorphic form contains at least 10% of the first polymorph. In a further embodiment, the solid form contains at least 20% of the first polymorph. Even further embodiments contain at least 30%, at least 40%, or at least 50% of the fist polymorph. One of skill in the art will recognize that many such combinations of several individual polymorphs in varying amounts are possible.

A. Polymorph Form I

Polymorphic Form I of Compound 1 can be produced by direct crystallization of Compound 1 from methanol and water by stirring at elevated temperature. Polymorphic Form I of Compound 1 is chemically stable at 80° C. and is stable at 40° C. under 75% relative humidity for at least 13 days. Polymorphic Form I of Compound 1 has an aqueous solubility of 179 μg/mL at pH 2 and 9 μg/mL at pH 6.5.

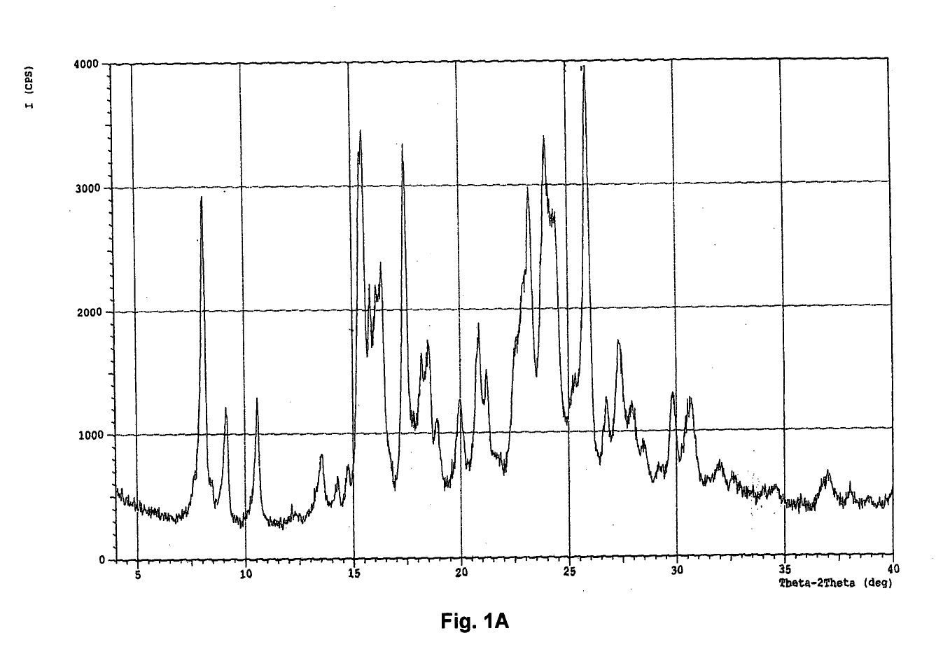

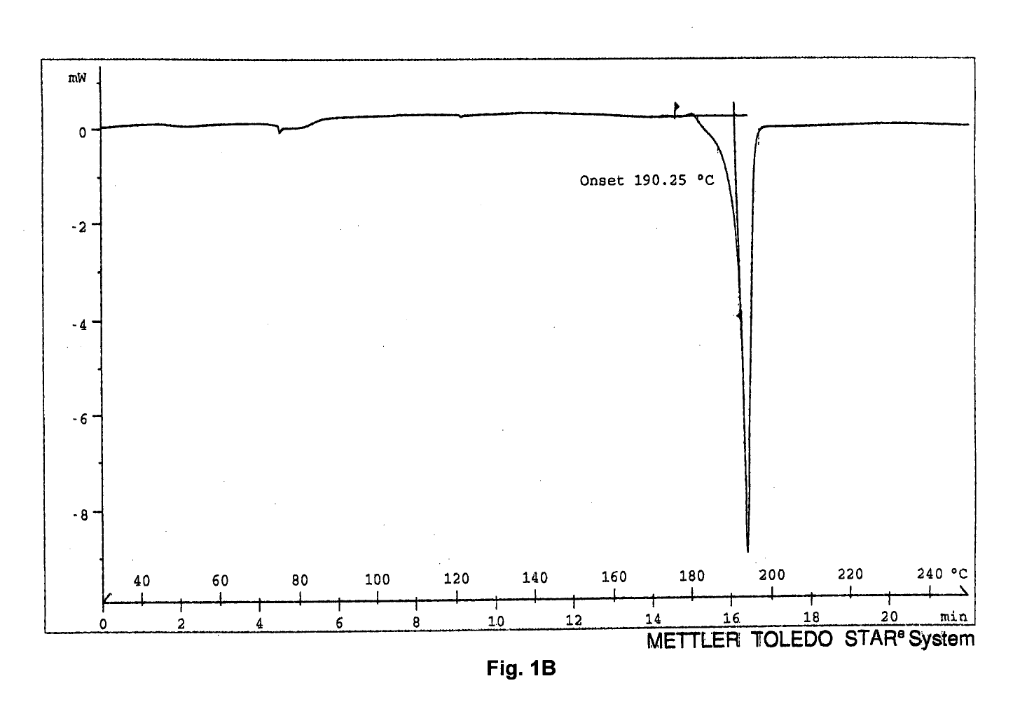

Form I is characterized by an X-ray powder diffraction pattern with peaks at the following approximate diffraction angles (2θ): 8.1, 9.1, 10.6, 15.4, 16.3, 17.4, 18.2, 18.5, 20.0, 20.8, 23.2, 24.0, 25.9, 27.4, and 29.8. FIG. 1A provides an X-ray powder diffraction pattern for Form I. The DSC thermogram for Form I, shown in FIG. 1B, indicates an endotherm onset at 183-190° C. at a scan rate of 10° C./min.

B. Polymorph Form II

Polymorphic Form II of Compound 1 is a hydrate. Polymorphic Form II of Compound 1 can be produced by exposing polymorphic Form I of Compound 1 to 93% relative humidity at room temperature for six days.

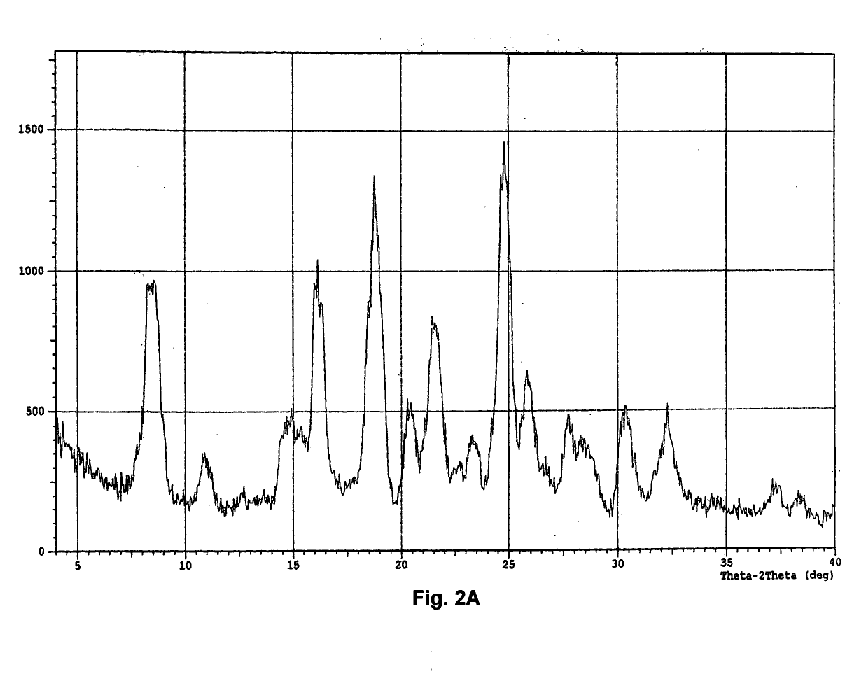

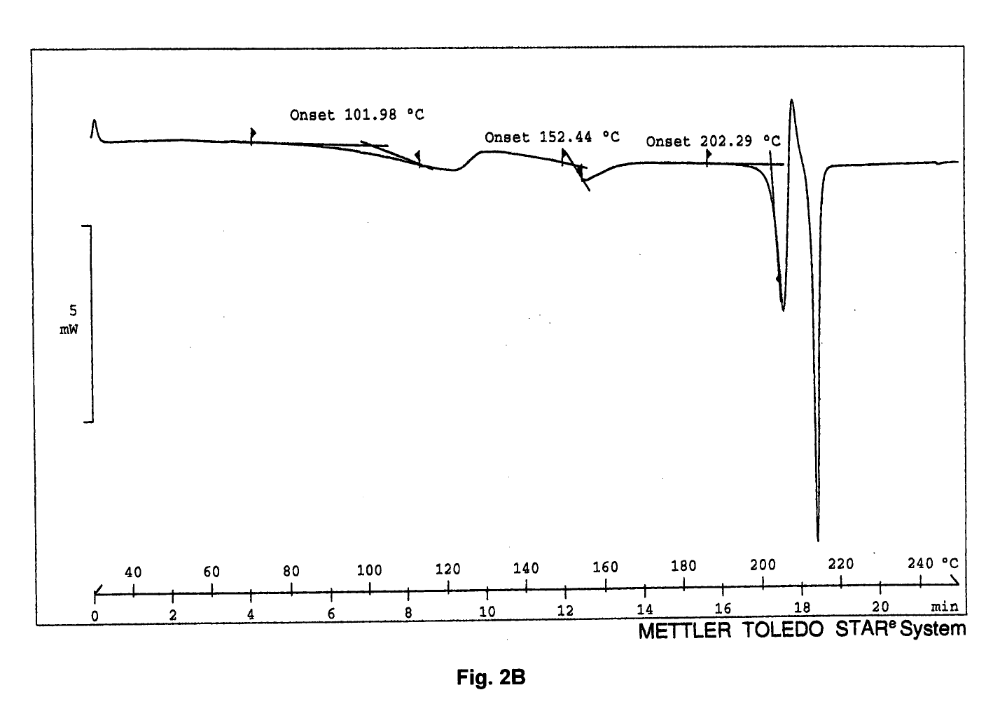

Form II is characterized by an X-ray powder diffraction pattern with peaks at the following approximate diffraction angles (2θ): 8.5, 10.9, 14.8, 16.2, 18.8, 21.5, 24.8, 25.9, 30.3, and 32.2. FIG. 2A provides an X-ray powder diffraction pattern for Form II. The DSC thermogram for Form II, shown in FIG. 2B, indicates an endotherm onset at 102, 152, and 202° C., followed by an exotherm at 206° C. and another exotherm at 210° C. at a scan rate of 10° C./min.

C. Polymorph Form III

Polymorphic Form III of Compound 1 can be produced by neutralizing a p-toluenesulfonic salt derivative of Compound 1 in ethylacetate with NaHCO₃ solution. Polymorphic Form III of Compound 1 is typically an ethyl acetate solvate.

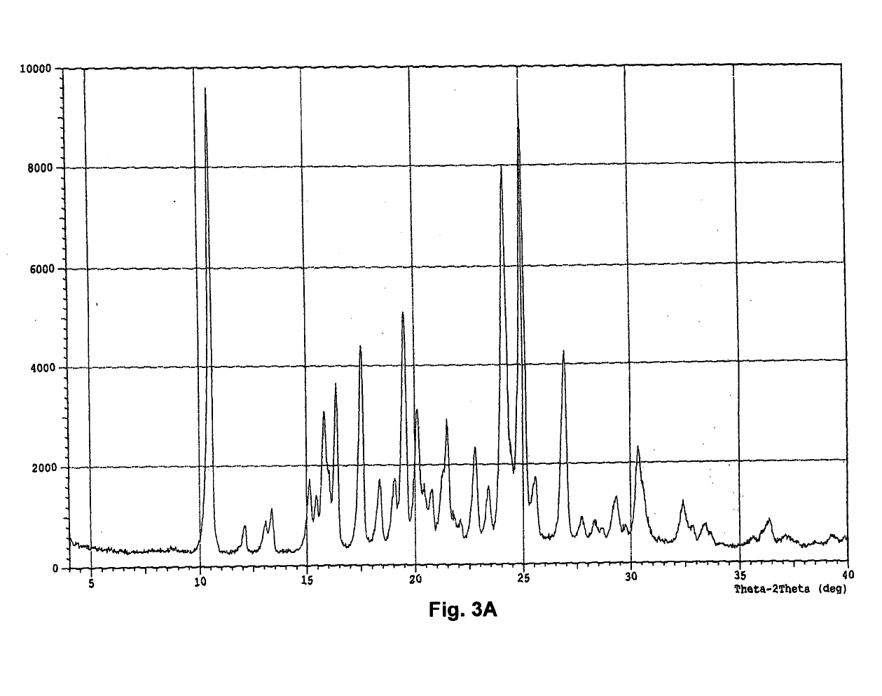

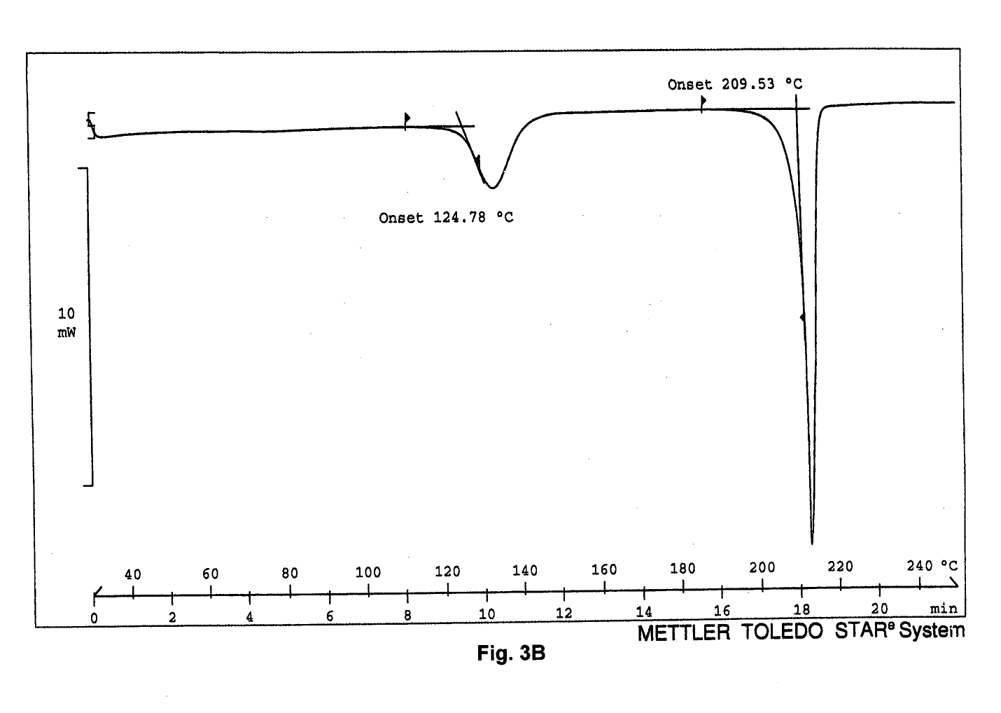

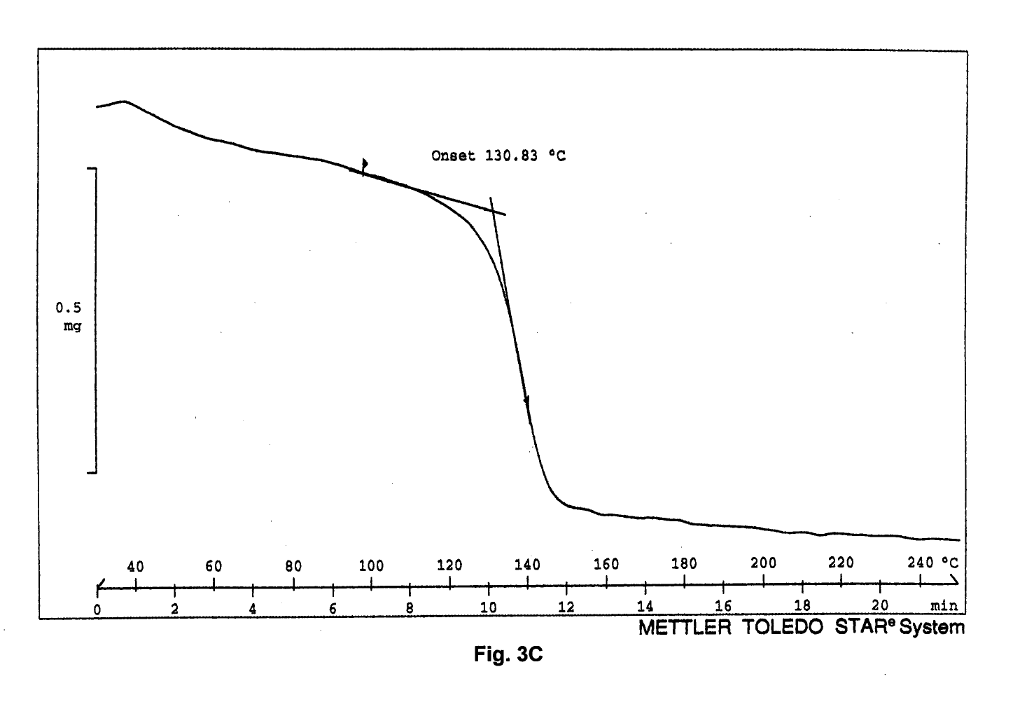

Form III is characterized by an X-ray diffraction pattern with peaks at the following approximate diffraction angles (2θ): 10.5, 13.0, 13.3, 15.8, 16.4, 17.5, 19.5, 20.1, 21.4, 21.7, 24.1, 25.0, and 26.9. FIG. 3A provides an X-ray powder diffraction pattern for Form III. The DSC thermogram for Form III, shown in FIG. 3B, indicates an endotherm onset at 125-129° C., followed by another endotherm at 210° C., at a scan rate of 10° C./min. Form III of Compound 1 has been further characterized by Thermal Gravimetric Analysis (TGA). FIG. 3C is a Thermal Gravimetric Analysis (TGA) profile of a sample of polymorphic Form III. A typical TGA thermogram of samples of polymorphic Form III of Compound 1 indicate desolvation. Loss of ethyl acetate is indicated by 10% sample weight loss at 125-129° C. at a scan rate of about 10° C./min.

D. Polymorph Form IV

Polymorphic Form IV of Compound 1 can be prepared with several different procedures: (i) direct desolvation of polymorphic Form III of Compound 1 in vacuo at 110-135° C.; (ii) via solid-state conversion of polymorphic Form III by slurrying polymorphic Form III in toluene or xylene at 110-140° C.; (iii) via recrystallization of Compound 1 from dichloromethane/methanol solution followed by slurrying the precipitate in toluene at 140° C.; (iv) via solid-state conversion of polymorphic Form VI by refluxing polymorphic Form VI as a toluene slurry at 140° C.; and (v) via precipitation of Compound 1 in PEG-400 solution with water. Aqueous solubility of polymorphic Form IV is about 550 μg/mL at about pH 1, about 157 μg/mL at about pH 2, about 6 μg/mL at about pH 4, about 2 μg/mL at about pH 6.5, and about 2 μg/mL at about pH 8.

Polymorphic Form IV is physically and chemically stable at 80° C. and at 40° C. under 75% relative humidity for at least 30 days. Polymorphic Form IV is believed to be the thermodynamically most stable form of Compound 1.

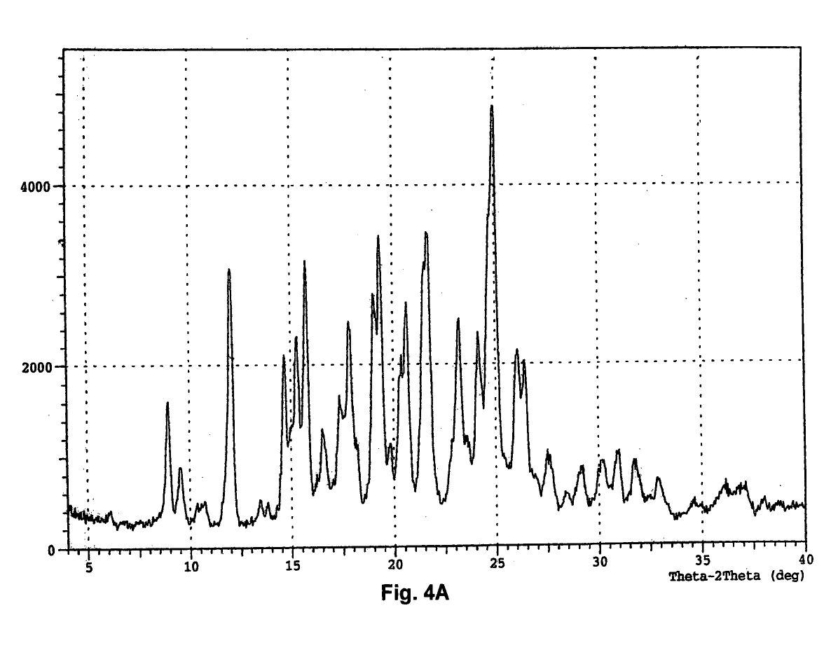

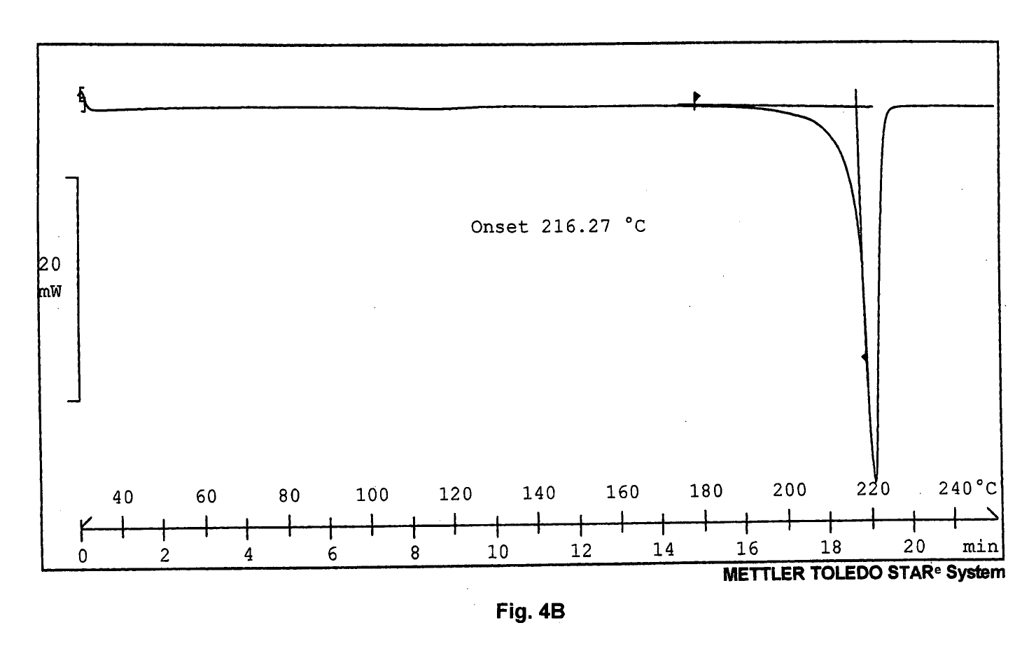

Form IV is further characterized by an X-ray diffraction pattern with peaks at the following approximate diffraction angles (2θ): 8.9, 12.0, 14.6, 15.2, 15.7, 17.8, 19.2, 20.5, 21.6, 23.2, 24.2, 24.8, 26.2, and 27.5. FIG. 4A provides an X-ray powder diffraction pattern for Form IV. The DSC thermogram for Form IV, shown in FIG. 4B, indicates an endotherm onset at 216° C. at a scan rate of 10° C./min.

E. Polymorph Form VI

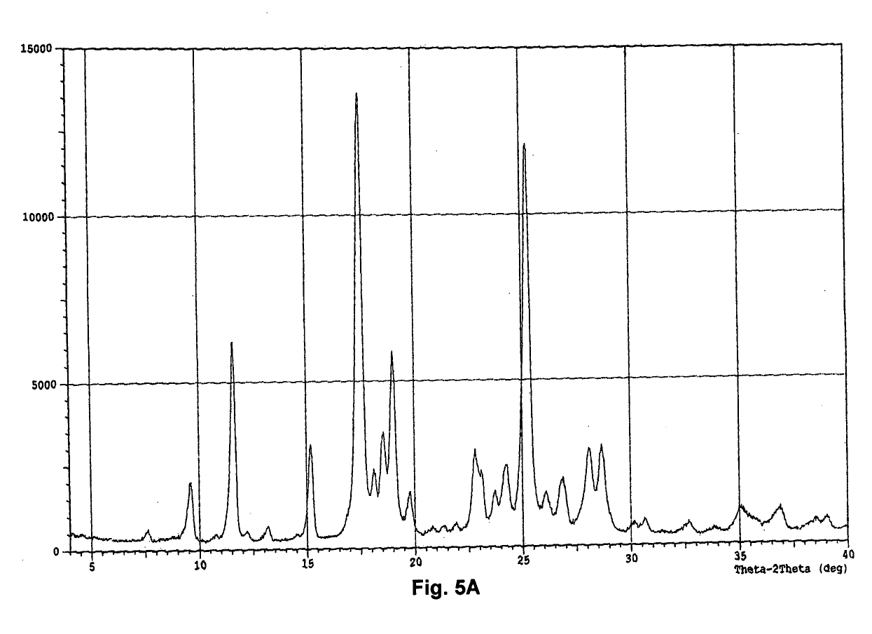

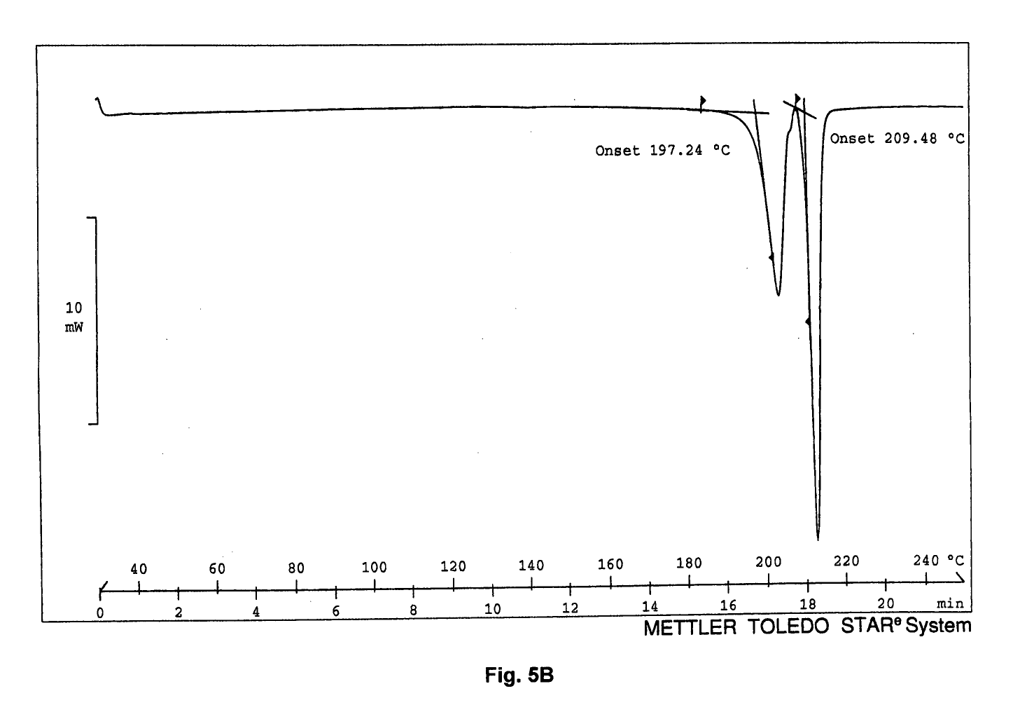

Polymorphic Form VI of Compound 1 can be prepared by direct crystallization of Compound 1 with ethanol in NaHCO₃ solution. Form VI is characterized by an X-ray diffraction pattern with peaks at the following approximate diffraction angles (2θ): 9.6, 11.6, 17.5, 18.1, 19.9, and 25.2. FIG. 5A provides an X-ray powder diffraction pattern of Form VI. The DSC thermogram for Form VI, shown in FIG. 5B, indicates an endotherm onset at 197° C. at a scan rate of 10° C./min.

F. Polymorph Form VII

Polymorphic Form VII of Compound 1 can be prepared by refluxing a suspension of polymorphic Form VI of Compound 1 in isopropanol, tetrahydrofuran, or methyl-tert-butyl ether.

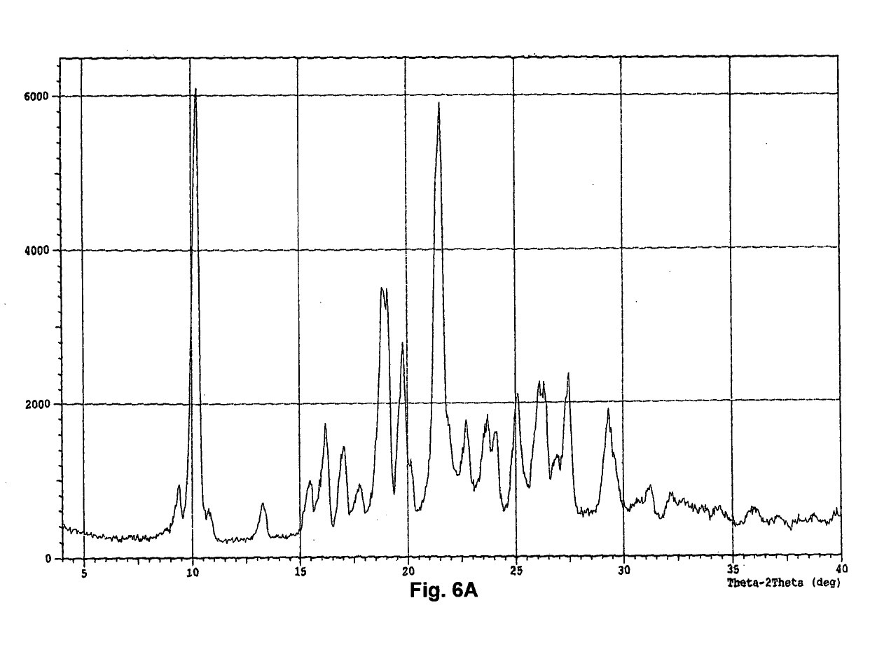

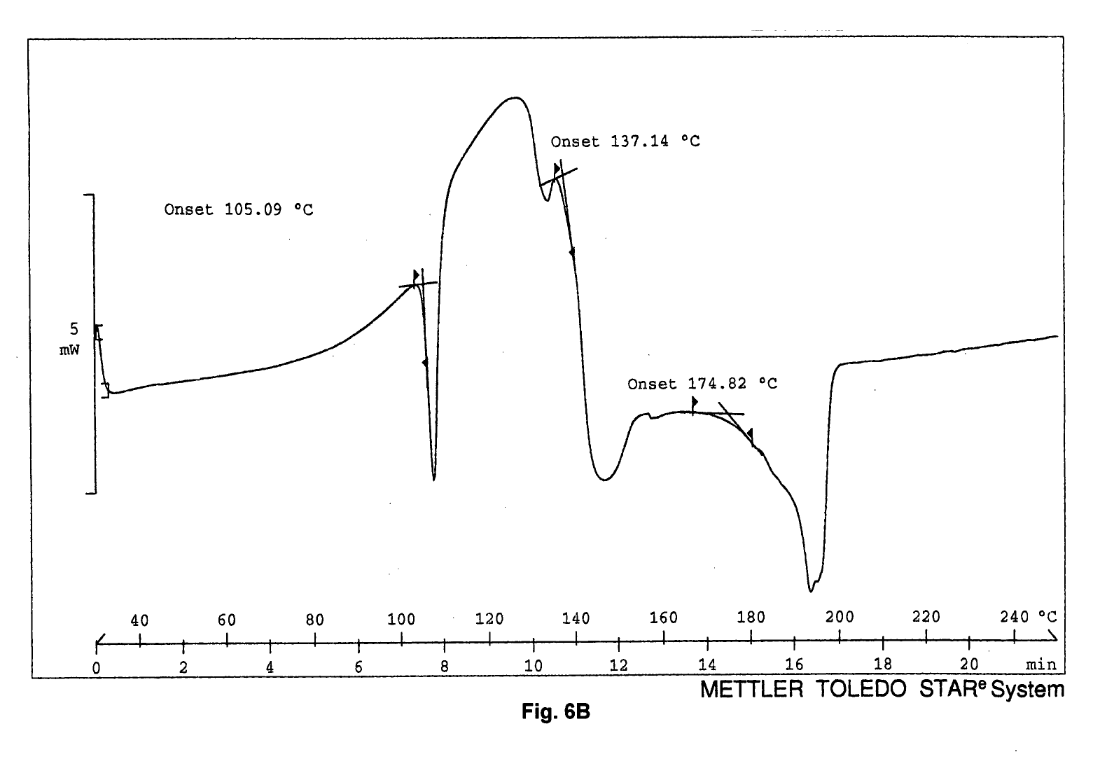

Form VII is characterized by an X-ray diffraction pattern with peaks at the following approximate diffraction angles (2θ): 9.4, 10.2, 16.2, 17.0, 18.9, 19.7, 21.5, 22.7, 23.6, 25.1, 26.2, 27.4, and 29.3. FIG. 6A provides an X-ray powder diffraction pattern from Form VII. The DSC thermogram for Form VII, shown in FIG. 6B, indicates an endotherm onset at 105° C., followed by an exothemm at 115° C., and then endotherms at 137 and 175° C., at a scan rate of 10° C./min.

G. Polymorph Form VIII

Polymorphic Form VIII of Compound 1 can be produced by refluxing a polymorphic Form VI suspension of Compound 1 in dioxane.

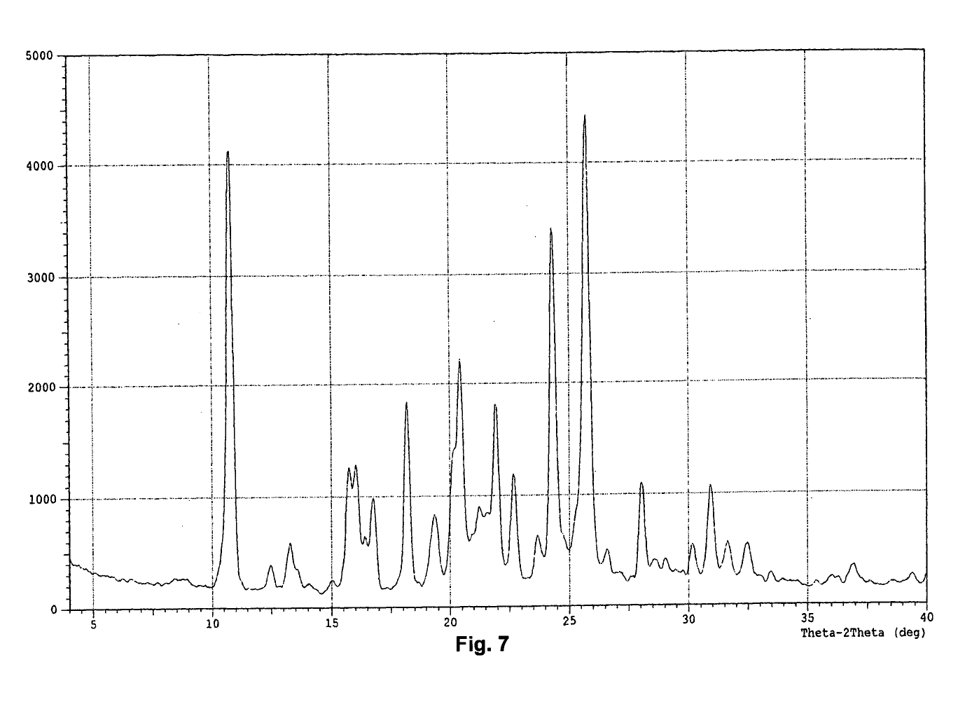

Form VIII is characterized by an X-ray diffraction pattern with peaks at the following approximate diffraction angles (2θ): 10.7, 15.5, 15.9, 20.6, 22.7, 24.6, 25.9, 26.3, and 32.0. FIG. 7 provides an X-ray powder diffraction pattern from Form VIII.

II. Pharmaceutical Compositions of the Invention

The active agents (i.e., the polymorphs, or solid forms comprising two or more such polymorphs, of Compound 1 described herein) of the invention may be formulated into pharmaceutical compositions suitable for mammalian medical use. Any suitable route of administration may be employed for providing a patient with an effective dosage of any of polymorphic Forms I, II, III, IV, VI, VII, and VIII of Compound 1, or a pharmaceutically acceptable salt thereof. For example, peroral or parenteral formulations and the like may be employed. Dosage forms include capsules, tablets, dispersions, suspensions and the like, e.g. enteric-coated capsules and/or tablets, capsules and/or tablets containing enteric-coated pellets of Compound 1, or a pharmaceutically acceptable salt thereof. In all dosage forms, polymorphic Form IV of Compound 1, or a pharmaceutically acceptable salt thereof can be admixtured with other suitable constituents. The compositions may be conveniently presented in unit dosage forms, and prepared by any methods known in the pharmaceutical arts. Pharmaceutical compositions of the invention comprise a therapeutically effective amount of the active agent and one or more inert, pharmaceutically acceptable carriers, and optionally any other therapeutic ingredients, stabilizers, or the like. The carrier(s) must be pharmaceutically acceptable in the sense of being compatible with the other ingredients of the formulation and not unduly deleterious to the recipient thereof. The compositions may further include diluents, buffers, binders, disintegrants, thickeners, lubricants, preservatives (including antioxidants), flavoring agents, taste-masking agents, inorganic salts (e.g., sodium chloride), antimicrobial agents (e.g., benzalkonium chloride), sweeteners, antistatic agents, surfactants (e.g., polysorbates such as “TWEEN 20” and “TWEEN 80′, and pluronics such as F68 and F88, available from BASF), sorbitan esters, lipids (e.g., phospholipids such as lecithin and other phosphatidylcholines, phosphatidylethanolamines, fatty acids and fatty esters, steroids (e.g., cholesterol)), and chelating agents (e.g., EDTA, zinc and other such suitable cations). Other pharmaceutical excipients and/or additives suitable for use in the compositions according to the invention are listed in Remington: The Science & Practice of Pharmacy, 19^{th} ed., Williams & Williams, (1995), and in the ‘Physician’s Desk Reference”, 52^{nd} ed., Medical Economics, Montvale, N.J. (1998), and in Handbook of Pharmaceutical Excipients, 3^{rd}. Ed., Ed. A. H. Kibbe, Pharmaceutical Press, 2000. The active agents of the invention may be formulated in compositions including those suitable for oral, rectal, topical, nasal, ophthalmic, or parenteral (including intraperitoneal, intravenous, subcutaneous, or intramuscular injection) administration.

The amount of the active agent in the formulation will vary depending upon a variety of factors, including dosage form, the condition to be treated, target patient population, and other considerations, and will generally be readily determined by one skilled in the art. A therapeutically effective amount will be an amount necessary to modulate, regulate, or inhibit a protein kinase. In practice, this will vary widely depending upon the particular active agent, the severity of the condition to be treated, the patient population, the stability of the formulation, and the like. Compositions will generally contain anywhere from about 0.001% by weight to about 99% by weight active agent, preferably from about 0.01% to about 5% by weight active agent, and more preferably from about 0.01% to 2% by weight active agent, and will also depend upon the relative amounts of excipients/additives contained in the composition.

A pharmaceutical composition of the invention is administered in conventional dosage form prepared by combining a therapeutically effective amount of an active agent as an active ingredient with one or more appropriate pharmaceutical carriers according to conventional procedures. These procedures may involve mixing, granulating and compressing or dissolving the ingredients as appropriate to the desired preparation.

The pharmaceutical carrier(s) employed may be either solid or liquid. Exemplary solid carriers include lactose, sucrose, talc, gelatin, agar, pectin, acacia, magnesium stearate, stearic acid and the like. Exemplary liquid carriers include syrup, peanut oil, olive oil, water and the like. Similarly, the carrier(s) may include time-delay or time-release materials known in the art, such as glyceryl monostearate or glyceryl distearate alone or with a wax, ethylcellulose, hydroxypropylmethylcellulose, methylmethacrylate and the like.

A variety of pharmaceutical forms can be employed. Thus, if a solid carrier is used, the preparation can be tableted, placed in a hard gelatin capsule in powder or pellet form or in the form of a troche or lozenge. The amount of solid carrier may vary, but generally will be from about 25 mg to about 1 g. If a liquid carrier is used, the preparation can be in the form of syrup, emulsion, soft gelatin capsule, sterile injectable solution or suspension in an ampoule or vial or non-aqueous liquid suspension.

To obtain a stable water-soluble dose form, a pharmaceutically acceptable salt of an active agent can be dissolved in an aqueous solution of an organic or inorganic acid, such as 0.3M solution of succinic acid or citric acid. If a soluble salt form is not available, the active agent may be dissolved in a suitable co-solvent or combinations of co-solvents. Examples of suitable co-solvents include, but are not limited to, alcohol, propylene glycol, polyethylene glycol 300, polysorbate 80, gylcerin and the like in concentrations ranging from 0-60% of the total volume. The composition may also be in the form of a solution of a salt form of the active agent in an appropriate aqueous vehicle such as water or isotonic saline or dextrose solution.

It will be appreciated that the actual dosages of the active agents used in the compositions of this invention will vary according to the particular crystalline form being used, the particular composition formulated, the mode of administration and the particular site, host and disease being treated. Those skilled in the art using conventional dosage-determination tests in view of the experimental data for an agent can ascertain optimal dosages for a given set of conditions. For oral administration, an exemplary daily dose generally employed is from about 0.001 to about 1000 mg/kg of body weight, more preferably from about 0.001 to about 50 mg/kg body weight, with courses of treatment repeated at appropriate intervals. Administration of prodrugs is typically dosed at weight levels that are chemically equivalent to the weight levels of the fully active form. In the practice of the invention, the most suitable route of administration as well as the magnitude of a therapeutic dose will depend on the nature and severity of the disease to be treated. The dose, and dose frequency, may also vary according to the age, body weight, and response of the individual patient. In general, a suitable oral dosage form may cover a dose range from 5 mg to 250 mg total daily dose, administered in one single dose or equally divided doses. A preferred dosage range is from 10 mg to 80 mg.

The compositions of the invention may be manufactured in manners generally known for preparing pharmaceutical compositions, e.g., using conventional techniques such as mixing, dissolving, granulating, dragee-making, levigating, emulsifying, encapsulating, entrapping or lyophilizing. Pharmaceutical compositions may be formulated in a conventional manner using one or more physiologically acceptable carriers, which may be selected from excipients and auxiliaries that facilitate processing of the active compounds into preparations that can be used pharmaceutically.

For oral administration, the compounds can be formulated readily by combining the active agents with pharmaceutically acceptable carriers known in the art. Such carriers enable the compounds of the invention to be formulated as tablets, pills, dragees, capsules, gels, syrups, slurries, suspensions and the like, for oral ingestion by a patient to be treated. Pharmaceutical preparations for oral use can be obtained using a solid excipient in admixture with the active agent, optionally grinding the resulting mixture, and processing the mixture of granules after adding suitable auxiliaries, if desired, to obtain tablets or dragee cores. Suitable excipients include: fillers such as sugars, including lactose, sucrose, mannitol, or sorbitol; and cellulose preparations, for example, maize starch, wheat starch, rice starch, potato starch, gelatin, gum, methyl cellulose, hydroxypropylmethyl-cellulose, sodium carboxymethylcellulose, or polyvinylpyrrolidone (PVP). If desired, disintegrating agents may be added, such as crosslinked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, polyvinyl pyrrolidone, Carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active agents.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate, and, optionally, stabilizers. In soft capsules, the active agents may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

For administration intranasally or by inhalation, the compounds for use according to the present invention can be conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of gelatin for use in an inhaler or insufflator and the like may be formulated containing a powder mix of the compound and a suitable powder base such as lactose or starch.

The active agents may be formulated for parenteral administration by injection, e.g., by bolus injection or continuous infusion. Formulations for injection may be presented in unit-dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include suspensions of the active agents and may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the active agents to allow for the preparation of highly concentrated solutions.

For administration to the eye, the active agent is delivered in a pharmaceutically acceptable ophthalmic vehicle such that the compound is maintained in contact with the ocular surface for a sufficient time period to allow the compound to penetrate the corneal and internal regions of the eye, including, for example, the anterior chamber, posterior chamber, vitreous body, aqueous humor, vitreous humor, cornea, iris/cilary, lens, choroid/retina and selera. The pharmaceutically acceptable ophthalmic vehicle may be, for example, an ointment, vegetable oil, or an encapsulating material. An active agent of the invention may also be injected directly into the vitreous and aqueous humor or subtenon.

Alternatively, the active ingredient may be in powder form for constitution with a suitable vehicle, e.g., sterile pyrogen-free water, before use. The compounds may also be formulated in rectal or vaginal compositions such as suppositories or retention enemas, e.g., containing conventional suppository bases such as cocoa buffer or other glycerides.

In addition to the formulations described above, the polymorphic forms may also be formulated as a depot preparation. Such long-acting formulations may be administered by implantation (for example, subcutaneously or intramuscularly) or by intramuscular injection. Thus, for example, the polymorphic forms may be formulated with suitable polymeric or hydrophobic materials (for example, as an emulsion in an acceptable oil) or ion-exchange resins, or as sparingly soluble derivatives, for example, as a sparingly soluble salt.

Additionally, the active agents may be delivered using a sustained-release system, such as semipermeable matrices of solid hydrophobic polymers containing the therapeutic agent. Various sustained-release materials have been established and are known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid- or gel-phase carriers or excipients. Examples of such carriers or excipients include calcium carbonate, calcium phosphate, sugars, starches, cellulose derivatives, gelatin, and polymers such as polyethylene glycols.

III. Methods of Using the Polymorphs of the Invention

The inventive polymorphic forms of Compound 1 are useful for mediating the activity of protein kinases. More particularly, the polymorphic forms are useful as anti-angiogenesis agents and as agents for modulating and/or inhibiting the activity of protein kinases, such as the activity associated with VEGF, FGF, CDK complexes, TEK, CHK1, LCK, FAK, and phosphorylase kinase among others, thus providing treatments for cancer or other diseases associated with cellular proliferation mediated by protein kinases in mammals, including humans.

Therapeutically effective amounts of the agents of the invention may be administered, typically in the form of a pharmaceutical composition, to treat diseases mediated by modulation or regulation of protein kinases. An “effective amount” is intended to mean that amount of an agent that, when administered to a mammal in need of such treatment, is sufficient to effect treatment for a disease mediated by the activity of one or more protein kinases, such as tyrosine kinases. Thus, a therapeutically effective amount of a compound of the invention is a quantity sufficient to modulate, regulate, or inhibit the activity of one or more protein kinases such that a disease condition that is mediated by that activity is reduced or alleviated. The effective amount of a given compound will vary depending upon factors such as the disease condition and its severity and the identity and condition (e.g., weight) of the mammal in need of treatment, but can nevertheless be routinely determined by one skilled in the art. “Treating” is intended to mean at least the mitigation of a disease condition in a mammal, such as a human, that is affected, at least in part, by the activity of one or more protein kinases, such as tyrosine kinases, and includes: preventing the disease condition from occurring in a mammal, particularly when the mammal is found to be predisposed to having the disease condition but has not yet been diagnosed as having it; modulating and/or inhibiting the disease condition; and/or alleviating the disease condition. Exemplary disease conditions include diabetic retinopathy, neovascular glaucoma, rheumatoid arthritis, psoriasis, age-related macular degeneration (AMD), and cancer (solid tumors).

The activity of the polymorphic forms of Compound 1 as modulators of protein kinase activity may be measured by any of the methods available to those skilled in the art, including in vivo and/or in vitro assays. Examples of suitable assays for activity measurements include those described in Parast C. et al., Biochemistry, 37, 16788-16801 (1998); Jeffrey et al., Nature, 376, 313-320 (1995); WIPO International Publication No. WO 97/34876; and WIPO International Publication No. WO 96/14843.

The present invention is also directed to combination therapeutic methods of treating a hyperproliferative disorder, or a disease condition mediated by VEGF activity, which comprises administering to a mammal in need thereof a therapeutically effective amount of a pharmaceutical composition which comprises any of the polymorphic forms, or pharmaceutical compositions discussed above, in combination with a therapeutically effective amount of one or more substances selected from anti-tumor agents, anti-angiogenesis agents, signal transduction inhibitors, and antiproliferative agents. Such substances include those disclosed in PCT Publication Nos. WO 00/38715, WO 00/38716, WO 00/38717, WO 00/38718, WO 00/38719, WO 00/38730, WO 00/38665, WO 00/37107 and WO 00/38786, the disclosures of which are incorporated herein by reference in their entireties.

Examples of anti-tumor agents include mitotic inhibitors, for example vinca alkaloid derivatives such as vinblastine vinorelbine, vindescine and vincristine; colchines allochochine, halichondrine, N-benzoyltrimethyl-methyl ether colchicinic acid, dolastatin 10, maystansine, rhizoxine, taxanes such as taxol (paclitaxel), docetaxel (Taxotere), 2′-N-[3-(dimethylamino)propyl]glutaramate (taxol derivative), thiocholchicine, trityl cysteine, teniposide, methotrexate, azathioprine, fluorouricil, cytocine arabinoside, 2′2′-difluorodeoxycytidine (gemcitabine), adriamycin and mitamycin. Alkylating agents, for example cis-platin, carboplatin oxiplatin, iproplatin, Ethyl ester of N-acetyl-DL-sarcosyl-L-leucine (Asaley or Asalex), 1,4-cyclohexadiene-1,4-dicarbamic acid, 2,5-bis(1-azirdinyl)-3,6-dioxo-, diethyl ester (diaziquone), 1,4-bis(methanesulfonyloxy)butane (bisulfan or leucosulfan) chlorozotocin, clomesone, cyanomorpholinodoxorubicin, cyclodisone, dianhydroglactitol, fluorodopan, hepsulfam, mitomycin C, hycantheonemitomycin C, mitozolamide, 1-(2-chloroethyl)-4-(3-chloropropyl)-piperazine dihydrochloride, piperazinedione, pipobroman, porfiromycin, spirohydantoin mustard, teroxirone, tetraplatin, thiotepa, triethylenemelamine, uracil nitrogen mustard, bis(3-mesyloxypropyl)amine hydrochloride, mitomycin, nitrosoureas agents such as cyclohexyl-chloroethylnitrosourea, methylcyclohexyl-chloroethylnitrosourea 1-(2-chloroethyl)-3-(2,6-dioxo-3-piperidyl)-1-nitroso-urea, bis(2-chloroethyl)nitrosourea, procarbazine, dacarbazine, nitrogen mustard-related compounds such as mechloroethamine, cyclophosphamide, ifosamide, melphalan, chlorambucil, estramustine sodium phosphate, strptozoin, and temozolamide. DNA anti-metabolites, for example 5-fluorouracil, cytosine arabinoside, hydroxyurea, 2-[(3hydroxy-2-pyrinodinyl)methylene]-hydrazinecarbothioamide, deoxyfluorouridine, 5-hydroxy-2-formylpyridine thiosemicarbazone, alpha-2′-deoxy-6-thioguanosine, aphidicolin glycinate, 5-azadeoxycytidine, beta-thioguanine deoxyriboside, cyclocytidine, guanazole, inosine glycodialdehyde, macbecin II, pyrazolimidazole, cladribine, pentostatin, thioguanine, mercaptopurine, bleomycin, 2-chlorodeoxyadenosine, inhibitors of thymidylate synthase such as raltitrexed and pemetrexed disodium, clofarabine, floxundine and fludarabine. DNA/RNA antimetabolites, for example, L-alanosine, 5-azacytidine, acivicin, aminopterin and derivatives thereof such as N-[2-chloro-5-[[(2,4-diamino-5-methyl-6-quinazolinyl)methyl]amino]benzoyl]-L-aspartic acid, N-[4-[[(2,4-diamino-5-ethyl-6-quinazolinyl)methyl]amino]benzoyl]-L-aspartic acid, N-[2-chloro-4-[[(2,4-diaminopteridinyl)methyl]amino]benzoyl]-L-aspartic acid, soluble Baker's antifol, dichloroallyl lawsone, brequinar, ftoraf, dihydro-5-azacytidine, methotrexate, N-(phosphonoacetyl)-L-aspartic acid tetrasodium salt, pyrazofuran, trimetrexate, plicamycin, actinomycin D, cryptophycin, and analogs such as cryptophycin-52 or, for example, one of the preferred anti-metabolites disclosed in European Patent Application No. 239362 such as N-(5-[N-(3,4-dihydro-2-methyl-4-oxoquinazolin-6-ylmethyl)-N-methylamino]-2-thenoyl)-L-glutamic acid; growth factor inhibitors; cell cycle inhibitors; intercalating antibiotics, for example adriamycin and bleomycin; proteins, for example interferon; and anti-hormones, for example anti-estrogens such as NolvadexL (tamoxifen) or, for example anti-androgens such as Casodex™ (4′-cyano-3-(4-fluorophenylsulphonyl)-2-hydroxy-2-methyl-3′-(trifluoromethyl)propionanilide). Such conjoint treatment may be achieved by way of the simultaneous, sequential or separate dosing of the individual components of the treatment.

Anti-angiogenesis agents include MMP-2 (matrix-metalloprotienase 2) inhibitors, MMP-9 (matrix-metalloprotienase 9) inhibitors, and COX-II (cyclooxygenase II) inhibitors. Examples of useful COX-II inhibitors include CELEBREX™ (alecoxib), valdecoxib, and rofecoxib. Examples of useful matrix metalloproteinase inhibitors are described in WO 96/33172 (published Oct. 24, 1996), WO 96/27583 (published Mar. 7, 1996), European Patent Application No. 97304971.1 (filed Jul. 8, 1997), European Patent Application No. 99308617.2 (filed Oct. 29, 1999), WO 98/07697 (published Feb. 26, 1998), WO 98/03516 (published Jan. 29, 1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606,046 (published Jul. 13, 1994), European Patent Publication 931,788 (published Jul. 28, 1999), WO 90/05719 (published May 331, 1990), WO 99/52910 (published Oct. 21, 1999), WO 99/52889 (published Oct. 21, 1999), WO 99/29667 (published Jun. 17, 1999), PCT International Application No. PCT/IB98/01113 (filed Jul. 21, 1998), European Patent Application No. 99302232.1 (filed Mar. 25, 1999), Great Britain patent application number 9912961.1 (filed Jun. 3, 1999), U.S. Provisional Application No. 60/148,464 (filed Aug. 12, 1999), U.S. Pat. No. 5,863,949 (issued Jan. 26, 1999), U.S. Pat. No. 5,861,510 (issued Jan. 19, 1999), and European Patent Publication 780,386 (published Jun. 25, 1997), all of which are herein incorporated by reference in their entirety. Preferred MMP-2 and MMP-9 inhibitors are those that have little or no activity inhibiting MMP-1. More preferred, are those that selectively inhibit MMP-2 and/or MMP-9 relative to the other matrix-metalloproteinases (i.e. MMP-1, MMP-3, MMP-4, MMP-5, MMP-6, MMP-7, MMP-8, MMP-10, MMP-11, MMP-12, and MMP-13).

Examples of MMP inhibitors include AG-3340, RO 32-3555, RS 13-0830, and the following compounds: 3-[[4-(4-fluoro-phenoxy)-benzenesulfonyl]-(1-hydroxycarbamoyl-cyclopentyl)-amino]-propionic acid; 3-exo-3-[4-(4-fluoro-phenoxy)-benzenesulfonylamino]-8-oxa-bicyclo[3.2.1]octane-3-carboxylic acid hydroxyamide; (2R, 3R) 1-[4-(2-chloro-4-fluoro-benzyloxy)-benzenesulfonyl]-3-hydroxy-3-methyl-piperidine-2-carboxylic acid hydroxyamide; 4-[4-(4-fluoro-phenoxy)-benzenesulfonylamino]-tetrahydro-pyran-4-carboxylic acid hydroxyamide; 3-[[4-(4-fluoro-phenoxy)-benzenesulfonyl]-(1-hydroxycarbamoyl-cyclobutyl)-amino]-propionic acid; 4-[4-(4-chloro-phenoxy)-benzenesulfonylamino]-tetrahydro-pyran-4-carboxylic acid hydroxyamide; 3-[4-(4-chloro-phenoxy)-benzenesulfonylamino]-tetrahydro-pyran-3-carboxylic acid hydroxyamide; (2R, 3R) 1-[4-(4-fluoro-2-methyl-benzyloxy)-benzenesulfonyl]-3-hydroxy-3-methyl-piperidine-2-carboxylic acid hydroxyamide; 3-[[4-(4-fluoro-phenoxy)-benzenesulfonyl]-(1-hydroxycarbamoyl-1-methyl-ethyl)-amino]-propionic acid; 3-[[4-(4-fluoro-phenoxy)-benzenesulfonyl]-(4-hydroxycarbamoyl-tetrahydro-pyran-4-yl)-amino]-propionic acid; 3-exo-3-[4-(4-chloro-phenoxy)-benzenesulfonylamino]-8-oxa-bicyclo[3.2.1]octane-3-carboxylic acid hydroxyamide; 3-endo-3-[4-(4-fluoro-phenoxy)-benzenesulfonylamino]-8-oxa-bicyclo[3.2.1]octane-3-carboxylic acid hydroxyamide; 3-[4-(4-fluoro-phenoxy)-benzenesulfonylamino]-tetrahydro-furan-3-carboxylic acid hydroxyamide; and pharmaceutically acceptable salts, solvates and hydrates thereof.

Examples of signal transduction inhibitors include agents that can inhibit EGFR (epidermal growth factor receptor) responses, such as EGFR antibodies, EGF antibodies, and molecules that are EGFR inhibitors; VEGF (vascular endothelial growth factor) inhibitors; and erbB2 receptor inhibitors, such as organic molecules or antibodies that bind to the erbB2 receptor, for example, HERCEPTIN™ (Genentech, Inc. of South San Francisco, Calif., USA).

EGFR inhibitors are described in, for example in WO 95/19970 (published Jul. 27, 1995), WO 98/14451 (published Apr. 9, 1998), WO 98/02434 (published Jan. 22, 1998), and U.S. Pat. No. 5,747,498 (issued May 5, 1998). EGFR-inhibiting agents include, but are not limited to, the monoclonal antibodies C225 and anti-EGFR 22Mab (ImClone Systems Incorporated of New York, N.Y., USA), the compounds ZD-1839 (AstraZeneca), BIBX-1382 (Boehringer Ingelheim), MDX-447 (Medarex Inc. of Annandale, N.J., USA), and OLX-103 (Merck & Co. of Whitehouse Station, N.J., USA), VRCTC-310 (Ventech Research) and EGF fusion toxin (Seragen Inc. of Hopkinton, Mass.).

VEGF inhibitors, for example SU-5416 and SU-6668 (Sugen Inc. of South San Francisco, Calif., USA), can also be combined or co-administered with the composition. VEGF inhibitors are described in, for example in WO 99/24440 (published May 20, 1999), PCT International Application PCT/IB99/00797 (filed May 3, 1999), in WO 95/21613 (published Aug. 17, 1995), WO 99/61422 (published Dec. 2, 1999), U.S. Pat. No. 5,834,504 (issued Nov. 10, 1998), WO 98/50356 (published Nov. 12, 1998), U.S. Pat. No. 5,883,113 (issued Mar. 16, 1999), U.S. Pat. No. 5,886,020 (issued Mar. 23, 1999), U.S. Pat. No. 5,792,783 (issued Aug. 11, 1998), WO 99/10349 (published Mar. 4, 1999), WO 97/32856 (published Sep. 12, 1997), WO 97/22596 (published Jun. 26, 1997), WO 98/54093 (published Dec. 3, 1998), WO 98/02438 (published Jan. 22, 1998), WO 99/16755 (published Apr. 8, 1999), and WO 98/02437 (published Jan. 22, 1998), all of which are herein incorporated by reference in their entirety. Other examples of some specific VEGF inhibitors are IM862 (Cytran Inc. of Kirkland, Wash., USA); anti-VEGF monoclonal antibody bevacizumab (Genentech, Inc. of South San Francisco, Calif.); and angiozyme, a synthetic ribozyme from Ribozyme (Boulder, Colo.) and Chiron (Emeryville, Calif.).

ErbB2 receptor inhibitors, such as GW-282974 (Glaxo Wellcome plc), and the monoclonal antibodies AR-209 (Aronex Pharmaceuticals Inc. of The Woodlands, Tex., USA) and 2B-1 (Chiron), may be administered in combination with the composition. Such erbB2 inhibitors include those described in WO 98/02434 (published Jan. 22, 1998), WO 99/35146 (published Jul. 15, 1999), WO 99/35132 (published Jul. 15, 1999), WO 98/02437 (published Jan. 22, 1998), WO 97/13760 (published Apr. 17, 1997), WO 95/19970 (published Jul. 27, 1995), U.S. Pat. No. 5,587,458 (issued Dec. 24, 1996), and U.S. Pat. No. 5,877,305 (issued Mar. 2, 1999), each of which is herein incorporated by reference in its entirety. ErbB2 receptor inhibitors useful in the present invention are also described in U.S. Provisional Application No. 60/117,341, filed Jan. 27, 1999, and in U.S. Provisional Application No. 60/117,346, filed Jan. 27, 1999, both of which are herein incorporated by reference in their entirety.

Other antiproliferative agents that may be used include inhibitors of the enzyme farnesyl protein transferase and inhibitors of the receptor tyrosine kinase PDGFr, including the compounds disclosed and claimed in the following U.S. patent application Ser. Nos. 09/221,946 (filed Dec. 28, 1998); 09/454,058 (filed Dec. 2, 1999); 09/501,163 (filed Feb. 9, 2000); 09/539,930 (filed Mar. 31, 2000); 09/202,796 (filed May 22, 1997); 09/384,339 (filed Aug. 26, 1999); and 09/383,755 (filed Aug. 26, 1999); and the compounds disclosed and claimed in the following U.S. provisional patent applications: 60/168,207 (filed Nov. 30, 1999); 60/170,119 (filed Dec. 10, 1999); 60/177,718 (filed Jan. 21, 2000); 60/168,217 (filed Nov. 30, 1999), and 60/200,834 (filed May 1, 2000). Each of the foregoing patent applications and provisional patent applications is herein incorporated by reference in their entirety.

Compositions of the invention can also be used with other agents useful in treating abnormal cell growth or cancer, including, but not limited to, agents capable of enhancing antitumor immune responses, such as CTLA4 (cytotoxic lymphocite antigen 4) antibodies, and other agents capable of blocking CTLA4; and anti-proliferative agents such as other farnesyl protein transferase inhibitors. Specific CTLA4 antibodies that can be used in the present invention include those described in U.S. Provisional Application 60/113,647 (filed Dec. 23, 1998) which is herein incorporated by reference in its entirety.

### EXAMPLES

The examples which follow will further illustrate the preparation of the distinct polymorphic forms of the invention, i.e. polymorphic Forms I, II, III, IV, VI, VII, and VIII of Compound 1, but are not intended to limit the scope of the invention as defined herein or as claimed below. Unless otherwise indicated, all temperatures are set forth in degrees Celsius and all parts and percentages are by weight. HPLC data was obtained using a Hewlett Packard HP-1100 HPLC.

### Example 1

### Preparation and Characterization of Polymorphic Form I of Compound 1

6-[2-(methylcarbamoyl)phenylsulfanyl]-3-E-[2-(pyridin-2-yl)ethenyl]indazole, (4.6 g) prepared for example according to Example 33(a) in U.S. Pat. No. 6,531,491 (hereby incorporated in its entirety by reference), was slurried in 50 mL methanol at 50° C. for 15 min. after which 50 mL water was then added. The slurry was stirred thoroughly and allowed to cool to room temperature. The solids were collected by filtration, washed with 50 mL water and then with 30 mL ethylacetate. The product was then dried under high vacuum. HPLC purity was greater than 99%.

FIG. 1A is an X-ray powder diffractogram of polymorphic Form I of Compound 1. Polymorphic Form I of Compound 1 was further characterized by differential scanning calorimetry. FIG. 1B is a differential scanning calorimetry (DSC) profile of a sample of polymorphic Form I of Compound 1. Samples of polymorphic Form I of Compound 1 displayed an endotherm with onset at 183-190° C. at a scan rate of about 10° C./min.

### Example 2

### Preparation and Characterization of Polymorphic Form II of Compound 1

Polymorphic Form II of Compound 1, which is a hydrate, was generated by placing polymorphic Form I of Compound 1 (37 mg) in a 93% relative humidity chamber at room temperature for six days. (HPLC purity >98.5%). FIG. 2A is an X-ray powder diffractogram of polymorph Form II of Compound 1. Polymorphic Form II of Compound 1 was further characterized by differential scanning calorimetry. FIG. 2B is a differential scanning calorimetry (DSC) profile of a sample of polymorphic Form II of Compound 1. Form II displayed endotherms with onset at 102, 152, and 202° C., followed by an exotherm at 206° C. and another exotherm at 210° C. at a scan rate of 10° C./min.

### Example 3

### Preparation and Characterization of Polymorphic Form III of Compound 1

Polymorphic Form III of Compound 1 was prepared by neutralizing a p-toluenesulfonic acid salt derivative of Compound 1 in ethyl acetate followed by drying under vacuum at 65° C. The p-toluene sulfonic acid salt of Compound 1 (421 g) was suspended in 1800 mL of 0.84 M NaHCO₃ and 1800 mL ethylacetate and stirred at 65° C. for 2 hrs. Solids were collected by filtration, washed with 1800 mL water and with 800 mL ethylacetate, and dried under lab vacuum at 50° C. overnight. Yield: 92% (HPLC purity was greater than 99%). Polymorphic Form III is an ethylacetate solvate.

FIG. 3A is an X-ray powder diffraction pattern of polymorphic Form III of Compound 1. Polymorphic Form III of Compound 1 was further characterized by differential scanning calorimetry. FIG. 3B is a differential scanning calorimetry (DSC) profile of a sample of polymorphic Form III of Compound 1. Samples of polymorphic Form III of Compound 1 displayed endotherms with onsets at 125-129° C., followed by another endotherm at 210° C. at a scan rate of about 10° C./min.

Polymorphic Form III was further characterized by Thermal Gravimetric Analysis (TGA). FIG. 3C is a Thermal Gravimetric Analysis (TGA) profile of a sample of polymorphic Form III. A typical TGA thermogram of samples of polymorphic Form III indicate desolvation. Loss of ethyl acetate is indicated by 10% sample weight loss at 125-129° C. at a scan rate of about 10° C./min.

### Example 4a

### Preparation and Characterization of Polymorphic Form IV of Compound I

Polymorphic Form IV of Compound 1 was prepared from polymorphic Form III of Compound 1. A sample of polymorphic Form III of Compound 1 (1.015 kg) was dissolved in 3 L of methanol and 5 L of acetic acid at 60° C. The solution was then filtered and concentrated by medium vacuum. 6 L of xylenes were added at 60° C. and then removed by full vacuum. 4 L of xylenes were added and then removed under full vacuum, followed by treatment with an additional 4 L of xylenes. Xylenes were then removed under full vacuum to yield polymorphic Form IV of Compound 1 in 92% Yield. HPLC analysis showed greater than 98.5% purity.

FIG. 4A is an X-ray powder diffraction pattern of polymorphic Form IV of Compound 1. Polymorphic Form IV of Compound 1 was further characterized by differential scanning calorimetry. FIG. 4B is a differential scanning calorimetry (DSC) profile of a sample of polymorphic Form IV. Samples of polymorphic Form IV of Compound 1 displayed an endotherm with onset at 216° C. at a scan rate of about 10° C./min.

### Example 4b

### Preparation and Characterization of Polymorphic Form IV of Compound 1

Following synthesis of Compound 1 where a palladium catalyst was used, the following procedure was carried out to remove the residual palladium and to crystallize Compound 1 in polymorphic Form IV.

To a 12 L 3-neck flask, equipped with a mechanical stirrer, was charged 160.20 g of Compound 1 and 1.6 L of DMA and 1.6 L of THF. After stirring for 20 minutes, the mixture became homogeneous. To the clear solution was charged 800.99 g of 10% cysteine-silica and the resulting mixture was allowed to stir at room temperature overnight. The mixture was filtered through “medium” sintered glass fritted funnel, and the cake was washed with a solution of 500 mL of DMA and 500 mL of THF. The cake was further washed with 2.0 L of THF and the filtrate was collected into a separate flask. The volatile parts in the latter filtrate was removed in vacuo and the residue was combined with the main filtrated. The combined filtrate was recharged back into the 12 L flask, followed by 800 g of 10% cysteine-silica. The flask was equipped with a mechanical stirrer and stirred over the weekend at room temperature. The mixture was filtered through “medium” sintered glass fritted funnel and the silica was washed with a mixture of solvents of 500 mL of DMA and 500 mL of THF, followed by 3.0 L of THF. The volatile parts in the filtrate were removed in vacuo and the remaining solution was transferred to a 22 L 3-neck flask and treated with 12 L of water (added over 20 minute period of time), a thick precipitate formed at this stage. After stirring overnight, the mixture was filtered and the cake was washed with 2.0 L of water and sucked dry.

The cake was charged to a 5 L 3-neck flask, followed by 1.6 L of THF and 160 mL of DMF. The flask was equipped with a mechanical stirrer, a reflux condenser and the mixture was heated at reflux for 8 hours. After cooling overnight, the mixture was filtered through sharkskin filter paper and sucked dry. The cake was charged to a 5 L 3-neck flask and 1.6 L of MeOH was added. The flask was equipped with a mechanical stirrer, a water condenser and the contents were heated at reflux for 6 hours. After cooling overnight, the mixture was filtered through sharkskin filter paper and sucked dry. The cake was dissolved into 1.6 L of HOAc with the assistance of gentle heating in the water bath of a rotary evaporator. The solution was filtered through #3 filter paper and the total volume of the filtrate was reduced to ˜500 mL in volume on the rotary evaporator at 60° C./60 mmHg. At this stage, the bulk of the mixture remained a yellow solution, a small amount of precipitate formed. To the flask was charged 500 mL of xylenes (precipitate formed) and the total volume was reduced to ˜500 mL in volume on the rotary evaporator at 60° C./60 mmHg. The process was repeated two additional times. After cooling, the mixture was filtered, the cake was washed with 500 mL of xylenes and sucked dry. The cake was transferred to a glass dish and further dried at 80° C./27 inch vacuum overnight. The cake was off-white in color and weighed 108.38 g, as was subsequently determined to be in a crystalline form of Form IV.

### Example 4c

### Preparation of Polymorphic Form IV of Compound 1

Polymorphic Form IV of compound 1 has also been prepared according to the following procedure. 2 kg of Compound 1 was charged to a 200 L reactor. Acetic acid (20 L) was then charged to the reactor via isolated vacuum. Note, a small amount of vacuum was used to avoid freezing the acetic acid during the charging process. Methanol (6 L) was then charged to the reactor, followed by heating to a temperature of 55 to 65° C. The contents of the reactor were then agitated for 30 to 45 minutes at 55 to 65° C. until a clear solution wass obtained. The contents of the reactor were then cooled to a temperature of 25 to 35° C. over a period of 1 to 2 hours. The contents were then filtered with a 14 or 18-inch Nutsche. A polypropylene 0.5 micrometer or less filter may be used as a back-up. The product rich filtrate was then collected within a clean polyethylene lined drum.

The reactor was then rinsed with acetic acid (10 L) and the rinse was forwarded onto the filter cake. The filtrate was then transferred to a 100 L reactor via a 0.2 micrometer polypropylene filter cartridge. The filtrate container was rinsed with acetic acid (10 L) and the rinse forwarded to the reactor via the 0.2 micrometer polypropylene filter cartridge. The contents were then concentrated to a final pot volume of 20 L, where the concentration was done under vacuum with a pot temperature between 60° C. and 70° C. The reactor was then cooled to a temperature of 25 to 35° C. Xylenes (20 L) were charged to the reactor via a 0.5 micrometer polypropylene filter. The reactor was then heated to a temperature of 60 to 70° C. The contents were then concentrated under vacuum to a final pot volume of 20 L.

The reactor was then cooled to a temperature of 25 to 35° C. Xylenes (20 L) were then charged to the reactor via the 0.5 micrometer polypropylene filter. The reactor was heated to a temperature of 60 to 70° C. The contents were then concentrated under vacuum to a final pot volume of 20 L. The reactor was then cooled to a temperature of 25 to 35° C. Xylenes (20 L) were then charged to the reactor via the 0.5 micrometer polypropylene filter. The reactor was then heated to a temperature of 60 to 70° C. The contents were then concentrated under vacuum to a final pot volume of 20 L. The reactor was then cooled to a temperature of 25 to 35° C. Xylenes (20 L) were again charged to the reactor via the 0.5 micrometer polypropylene filter. When solids were present on the upper wall of the reactor, they were scraped down through the handhole.

The reactor was then heated to a temperature of 60 to 70° C. The contents were then concentrated under vacuum to a final pot volume of 20 L. The reactor was then cooled to a temperature of 20 to 30° C. Samples were then submitted for DSC and XRD to confirm the formation of the desired form (form IV). Additional xylene evaporation (as described above) may be required to obtain the desired form.

The contents were then filtered through a product filter. Note, the filter should be Speck Free and dressed with a polypropylene cloth. The reactor was then charged with xylenes (20 L) via the 0.5 micrometer polypropylene filter and the rinse transferred onto the filter cake. The reactor was then charged with n-Heptane (20 L) via the 0.5 micrometer polypropylene filter and the rinse transferred onto the filter cake. The filter cake was then transferred from the Nutsche filter to a Tray dryer (e.g. porous polypropylene dryer tray covers). Drying occurred under full vacuum conditions at 40 to 50° C. with a slight (3-6 SCFH) nitrogen bleed for a minimum of 24 hours. Note, the time frame is open ended since an LOD of less than 0.5% must be achieved. 1.80 Kg of off-white solid was obtained, with a yield of 90%.

### Example 5

### Preparation and Characterization of Polymorphic Form VI of Compound 1

Polymorphic Fom III of Compound 1, (2 g) was suspended in 15 mL ethanol. 4 g of para-toluenesulfonic acid monohydrate was added and the mixture heated to 82° C. for 14 hr. After cooling to room temperature, 25 mL of saturated NaHCO₃ solution was added and the suspension stirred for 2 hr. Solids were collected by filtration, washed with 50 mL water and dried under lab vacuum at 45° C. overnight (HPLC purity>99%).

FIG. 5A is an X-ray powder diffraction pattern of polymorphic Form VI of Compound 1. Polymorphic Form VI of Compound 1 was further characterized by differential scanning calorimetry. FIG. 5B is a differential scanning calorimetry (DSC) profile of a sample of polymorphic Form VI of Compound 1. Form VI displayed an endotherm with onset at about 197° C. followed by another endotherm at about 209° C. at a scan rate of about 10° C./min.

### Example 6

### Preparation and Characterization of Polymorphic Form VII of Compound 1

Polymorphic Form VI of Compound 1 (102 mg) was suspended in 20 mL isopropyl alcohol, refluxed for 30 min, and cooled to room temperature. Solids were collected by filtration, washed with isopropyl alcohol, and dried under vacuum. Polymorphic Form VII of Compound 1 is an isopropanol solvate.

FIG. 6A shows an X-ray powder diffraction pattern of polymorphic Form VII of Compound 1. Form VII of Compound 1 was further characterized by differential scanning calorimetry. FIG. 6B is a differential scanning calorimetry (DSC) profile of a sample of polymorphic Form VII of Compound 1. Typical profiles are sample-dependent. One sample isolated from refluxing THF showed an endotherm at 105° C. followed by an exotherm at 115° C., and then endotherms at 137 and 175° C., at a scan rate of about 10° C./min.

### Example 7

### Preparation and Characterization of Polymorphic Form VIII of Compound 1

Polymorphic Form VII of Compound 1 was dissolved in a minimal amount of refluxing dioxane at about 100° C. and then allowed to cool to room temperature overnight. Large yellow crystals were collected by filtration, washed with dioxane, and dried under vacuum. Polymorphic Form VIII of Compound 1 is a dioxane solvate. FIG. 7 shows an X-ray powder diffraction pattern of polymorphic Form VIII of Compound 1.

### Example 8

### Use of Polymorphic Form IV In Tablet Formulations

Povidone (4% w/w) is dissolved in water (5 times, w/w) to form a solution for granulation. Polymorphic Form IV of Compound 1 (37%, w/w), prepared as in Example 4, is combined with lactose (25%, w/w), corn starch (16%, w/w), and a portion of croscarmellose sodium (2%, w/w) in a high sheer granulator. The mixture is dry blended, and then granulated with the povidone solution. The granulation is first welted for 2 minutes and dried at 60° C. to a loss-on-drying value of 5% or less. The material is dry milled with screen size 045 R. The milled material is blended with the remaining croscarmellose sodium (3%, w/w) and microcrystalline cellulose (12%, w/w). The blended mixture is blended again with magnesium stearate (1%, w/w). The mixture is compressed on a tablet compression equipment to produce tablets with containing 160 mg of Compound 1 per tablet.

### Example 9

### Generation of Acid Salts of Compound 1

Salt screening was performed for Compound 1 to improve its aqueous solubility. Compound 1 was added to seven different 100 mM acid solutions and stirred for 14 days to generate, in situ, seven acid salt forms of Compound 1. The seven acids used were as follows: methane sulfonic acid; sulfuric acid; hydrochloric acid; phosphoric acid; hydrobromic acid; maleic acid; and benzene sulfonic acid. For each of these different acids, 20 mg of Compound 1 was stirred in a sealed vial in the dark with 1.6 mL of a 100 mM solution of the acid of interest. To ensure that the maximum solubility level was reached, the samples were checked periodically to ensure that excess solid was present. After 8 days 400 μL of the mixture was removed and centrifuged at 14,000 rpm for 5 minutes. 100 μL of the supernatant was then removed, diluted with 900 μL of a 1:1 mixture of acetonitrile/methanol, and then analyzed by HPLC. A second set of data was gathered 14 days following the start of the experiment to observe any long-term changes in the solubility. Samples at 14 days were prepared following the same procedure described for the study at 8 days. The HPLC analysis was performed using a Primesphere column, C₁₈, 5 μm, 150×4.6 mm, with a flow rate of 1.5 mL/min and an injection volume of 10 μL. The table below summarizes the solubility of the seven different salt forms of Compound 1 that were formed over 2 weeks. In general the solubility values showed only small changes from 8 to 14 days.

| | Solubility 8 days | Solubility 14 days |
|---|---|---|
| Salt | (μg/mL) | (μg/mL) |
| Methane sulfonic acid | 1970 | 1835 |
| Suifuric acid | 601 | 603 |
| Hydrochloric acid | 576 | 549 |
| Phosphoric acid | 295 | 292 |
| Hydrobromic acid | 277 | 220 |
| Maleic acid | 69 | 68 |
| Benzene sulfonic acid | 10 | 11 |

The salt forms of Compound 1 that showed the highest solubility (methane sulfonic acid, sulfuric acid, and hydrochloric acid, were further characterized. Approximately 30 mg of each slat was placed in a vial in a chamter at room temperature and 93% relative humidity. After 6 days the percent weight of water absorbed, the X-ray powder diffraction pattern, and the differential scanning calorimetry data were obtained. Two polymorphs were observed for the hydrochloric acid salt (Forms I and II), three polymorphs were observed for the methane sulfonic acid salt (Forms I, II, and III), and three polymorphs were observed for the sulfuric acid salt (I, II, and III). These polymorphic forms were further analyzed with regard to stability to high intensity light. Approximately 0.4 mg of each salt was weighed into an HPLC vial. This was repeated five times total for each salt to give four samples and one standard. The samples were placed in a high intensity light chamber and irradiated for 0, 1, 2, and 6 hours. 1 mL of acetonitrile and 1 mL of methanol were added to dissolve each standard and sample prior to HPLC analysis. Except for Form II of the sulfuric acid salt, all samples degraded significantly (14%-97%) upon exposure to high intensity light.

### Example 10

### Human Metabolites of Compound 1

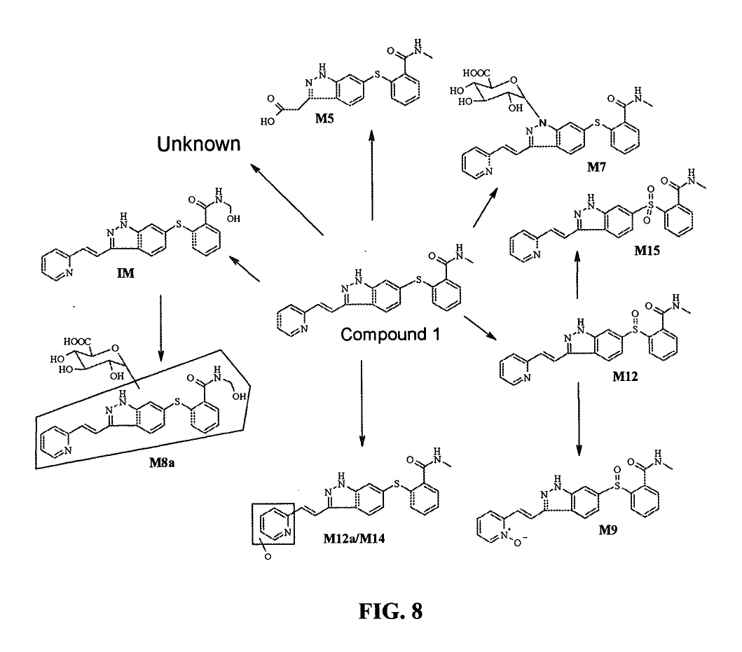

Compound 1 undergoes extensive metabolism to a variety of metabolites in humans, as shown in FIG. 8. The chemical structures of three oxygenated metabolites, M12 (the sulfoxide of Compound 1), M15 (the sulfone of Compound 1) and M9 (a mixed sulfoxidized/N-oxidized product of Compound 1), were confirmed based on the comparison in chromatographic retention times and mass spectra of the in-vivo metabolites to their authentic reference standards. The chemical structure of the glucuronide (M7) of Compound 1 was confirmed by the isolation of the metabolite followed by NMR determination. The metabolite M5 demonstrated an [M+H]⁺ ion at m/z 342. Interpretation of the MS² and MS³ product ion mass spectra of M5 suggested that M5 was a depyridinyl carboxylic acid of Compound 1. The proposed structures (or elemental compositions) of M5 and its major fragment ions (m/z 342, 311, 265, and 237) were all highly consistent with the elemental compositions determined by accurate mass measurement (with mass measurement accuracy ≦1.2 ppm for all). The definitive structures of metabolites M8a, M12a and M14 are currently unknown.

We claim:

1. A crystalline form of 6-[2-(methylcarbamoyl)phenylsulfanyl]-3-E-[2-(pyridin-2-yl)ethenyl]indazole, represented by Formula 1 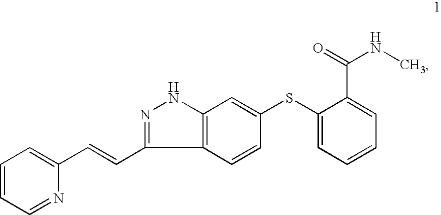
or a pharmaceutically acceptable salt thereof.

2. The crystalline form of claim 1, wherein the crystalline form is selected from the group consisting of polymorph Form I, Form II, Form III, Form IV, Form VI, Form VII, and Form VIII.

3. The crystalline form of claim 1, wherein the crystalline form is a polymorph of Form IV.

4. The crystalline form of claim 1, wherein the crystalline form has a powder X-ray diffraction pattern comprising peaks at diffraction angles (2θ) of 8.9±0.1 and 15.7±0.1.

5. The crystalline form of claim 1, wherein the crystalline form has a powder X-ray diffraction pattern comprising peaks at diffraction angles (2θ) of 8.9±0.1, 14.6±0.1, 15.7±0.1, and 19.2±0.1.

6. The crystalline form of claim 1, wherein the crystalline form has a powder X-ray diffraction pattern comprising peaks at diffraction angles (2θ) essentially the same as shown in FIG. 4A.

7. A pharmaceutical composition comprising the crystalline form of any of claims 1 to 6.

8. A method of treating a mammalian disease condition mediated by protein kinase activity, comprising administering to a mammal in need thereof a therapeutically effective amount of the pharmaceutical composition of claim 7.

9. The method according to claim 8, wherein the mammalian disease condition is associated with tumor growth, cell proliferation, or angiogenesis.
